(19) United States
(12) Patent Application Publication
(10) Pub. No.: US 2015/0064241 A1
(43) Pub. Date: Mar. 5, 2015

(54) Delivery of Functionalized Particles

(71) Applicant: Google Inc., Mountain View, CA (US)

(72) Inventor: Andrew Jason Conrad, Mountain View, CA (US)

(73) Assignee: Google Inc., Mountain View, CA (US)

(21) Appl. No.: 14/018,605

(22) Filed: Sep. 5, 2013

(51) Int. Cl. A61K 9/48

(52) U.S. Cl. CPC A61K 9/48
USPC 424/451

(57) ABSTRACT

A device includes a capsule sized to pass through a lumen of a gastrointestinal tract, a plurality of functionalized particles disposed within the capsule, one or more tissue penetrating members configured to puncture a wall of the lumen of the intestinal tract; and an actuator having a first configuration and a second configuration. The actuator is configured to retain the plurality of functionalized particles within the capsule in the first configuration. The actuator is further configured to advance the plurality of functionalized particles from the capsule into a wall of the lumen of the gastrointestinal tract via the one or more tissue penetrating members by the actuator transitioning from the first configuration to the second configuration. Systems including the device and methods of delivering functionalized particles to the body are also provided.

### BACKGROUND

Unless otherwise indicated herein, the materials described in this section are not prior art to the claims in this application and are not admitted to be prior art by inclusion in this section.

A number of scientific methods have been developed in the medical field to evaluate physiological conditions of a person by detecting and/or measuring one or more analytes in a person's blood or other bodily fluids. The one or more analytes could be any analytes that, when present in or absent from the blood, or present at a particular concentration or range of concentrations, may be indicative of a medical condition or health state of the person. The one or more analytes could include enzymes, reagents, hormones, proteins, cells or other molecules, such as carbohydrates, e.g., glucose.

In a typical scenario, a person's blood is drawn and either sent to a lab or input into a handheld testing device, such as a glucose meter, where one or more tests are performed to measure various analyte levels and parameters in the blood. For most people, the blood tests are infrequent, and an abnormal analyte level indicative of a medical condition may not be identified until the next blood test is performed. Even in the case of relatively frequent blood testing, such as may be found with those with diabetes, who regularly draw blood to test for blood glucose concentration, those blood tests are typically performed when the user is awake, although the blood glucose levels (and potential variations in such levels) occurring during the night could provide important information to assist a physician in assessing that person's medical condition. Further, most known methods of analyte detection and analysis require the collection of blood or other bodily fluid samples, which may be inconvenient, invasive and require significant patient compliance.

Methods for introduction of imaging, therapeutic or medicinal agents into the body, for the treatment or analysis of medical conditions include oral, intravenous, intramuscular, subcutaneous, transmucosal and topical delivery. Some of these methods may not be applicable for the delivery of all agents or substances. For example, some proteins, antibodies, peptides, vaccines and gene-based drugs cannot be given via traditional oral delivery methods due to a number of reasons, including: poor oral toleration, with complications including gastric irritation and bleeding; breakdown/degradation of the drug compounds in the stomach; and poor, slow, erratic or inefficient absorption of the drug due to molecular size and charge issues. Conventional alternative drug delivery methods such as intravenous and intramuscular delivery have a number of drawbacks including pain and risk of infection from a needle stick, requirements for the use of sterile technique and the requirement and associated risks of maintaining an IV line in a patient for an extended period of time. While other drug delivery approaches have been employed such as implantable drug delivery pumps, these approaches require the semi-permanent implantation of a device and can still have many of the limitations of IV delivery.

### SUMMARY

Some embodiments of the present disclosure provide a device comprising: (1) a capsule sized to pass through a lumen of a gastrointestinal tract; (2) a plurality of functionalized particles disposed within the capsule; (3) one or more tissue penetrating members configured to puncture a wall of the lumen of the intestinal tract; and (4) an actuator having a first configuration and a second configuration, wherein the actuator is configured to retain the plurality of functionalized particles within the capsule in the first configuration, and wherein the actuator is configured to advance the plurality of functionalized particles from the capsule into a wall of the lumen of the gastrointestinal tract via the one or more tissue penetrating members by the actuator transitioning from the first configuration to the second configuration.

Some embodiments of the present disclosure provide a method, including: (i) ingesting a device having: (1) a capsule containing a plurality of functionalized particles, wherein the capsule is sized to pass through a lumen of a gastrointestinal tract; and (2) one or more tissue penetrating members configured to puncture a wall of the lumen of the gastrointestinal tract, each of the one or more tissue penetrating members having a respective penetrating-member lumen and penetrating-member exit through which the functionalized particles can pass; and (ii) delivering, via the one or more tissue penetrating members, at least a portion of the plurality of functionalized particles into the wall of the lumen of the gastrointestinal tract.

Embodiments of the present disclosure further provide a system, comprising: (1) a swallowable device comprising: (i) a capsule sized to pass through a lumen of a gastrointestinal tract; (ii) one or more tissue penetrating members configured to puncture a wall of the lumen of the intestinal tract; and (iii) an actuator having a first configuration and a second configuration, wherein the actuator is configured to retain the plurality of functionalized particles within the capsule in the first configuration, and wherein the actuator is configured to advance the plurality of functionalized particles from the capsule into a wall of the lumen of the gastrointestinal tract via the one or more tissue penetrating members by the actuator transitioning from the first configuration to the second configuration; (2) a plurality of functionalized particles disposed within the capsule, the functionalized particles configured to interact with one or more target analytes present in blood in a lumen of subsurface vasculature; and (3) a detector configured to detect an analyte response signal transmitted from the portion of subsurface vasculature, wherein the analyte response signal is related to the interaction of the one or more target analytes with the functionalized particles.

Embodiments of the present invention further provide a method, including, loading a plurality of functionalized particles into a device having: (a) a capsule sized to pass through a lumen of a gastrointestinal tract; (ii) one or more tissue penetrating members configured to puncture a wall of the lumen of the intestinal tract, each of the tissue penetrating members having a respective penetrating-member exit; and (iii) an actuator having a first configuration and a second configuration, wherein the actuator is configured to retain the plurality of functionalized particles within the capsule in the first configuration, and wherein the actuator is configured to advance the plurality of functionalized particles from the capsule into a wall of the lumen of the gastrointestinal tract via the one or more tissue penetrating members by the actuator transitioning from the first configuration to the second configuration.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 is a perspective view of an embodiment of a swallowable delivery device.

FIGS. 2A-2D are side views of embodiments of tissue penetrating members for use in a swallowable delivery device.

FIG. 3 is a side view of an embodiment of a tissue penetrating member for use in a swallowable delivery device.

FIG. 4 is a side sectional view of an embodiment of a swallowable delivery device.

FIG. 5 is a side sectional view of an embodiment of a swallowable delivery device.

FIG. 6 is a side sectional view of an embodiment of a tissue penetrating member for use in a swallowable delivery device.

FIGS. 7A-7D are side sectional views of an embodiment of a swallowable delivery device, shown in a lumen of the intestinal tract.

FIG. 7E is a perspective view of an embodiment of a portion of an actuator and a tissue penetrating member for use in a swallowable delivery device.

FIG. 8 is a side sectional view of an embodiment of a tissue penetrating member for use in a swallowable delivery device.

FIG. 9 is an embodiment of a release for use in a swallowable delivery device.

FIG. 10 is an embodiment of a release for use in a swallowable delivery device.

FIGS. 11A-11C are side sectional views of an embodiment of a swallowable delivery device, shown in a lumen of the intestinal tract.

FIG. 12A is a front sectional view of an embodiment of a swallowable delivery device.

FIG. 12B is a side sectional view of an embodiment of a swallowable delivery device.

FIGS. 13A-13B are side sectional views of an embodiment of a swallowable delivery device, shown in a lumen of the intestinal tract.

FIGS. 14A-14D are side sectional views of an embodiment of a swallowable delivery device, shown in a lumen of the intestinal tract.

FIG. 15 is a view of an embodiment of a system including a swallowable delivery device.

FIG. 16 is a perspective view of an example wearable device for detecting and measuring a plurality of physiological parameters.

FIG. 17A is a perspective top view of an example wrist-mounted device, when mounted on a wearer's wrist.

FIG. 17B is a perspective bottom view of an example wrist-mounted device shown in FIG. 17A, when mounted on a wearer's wrist.

FIG. 18 is a block diagram of an example system that includes a plurality of wrist mounted devices in communication with a server.

FIG. 19 is a block diagram of an example method of delivering functionalized particles.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

### I. OVERVIEW

Quantitative and qualitative information regarding physiological parameters relating to the health of the person may be obtained by noninvasively measuring, with a wearable device mounted to the body, one or more analytes in blood circulating in subsurface vasculature. The one or more analytes could be any analytes that, when present in the blood at a particular concentration or range of concentrations, may be indicative of a medical condition or health of the person wearing the device. For example, the one or more analytes could include enzymes, hormones, proteins, or other molecules.

In an example embodiment, the wearable device obtains at least some of the health-related information by detecting the binding of a clinically-relevant analyte to functionalized particles, such as microparticles or nanoparticles, introduced into a lumen of the subsurface vasculature. The particles may be made of an inert material, such as polystyrene, and can have a diameter that is less than about 20 micrometers. In some embodiments, the particles have a diameter on the order of about 10 nm to 1 μm. In further embodiments, small particles on the order of 10-100 nm in diameter may be assembled to form a larger “clusters” or “assemblies on the order of 1-10 micrometers. Those of skill in the art will understand a “particle” in its broadest sense and that it may take the form of any fabricated material, a molecule, cryptophan, a virus, a phage, etc. Further, a particle may be of any shape, for example, spheres, rods, non-symmetrical shapes, etc. In some examples, the particles may be magnetic and can be formed from a paramagnetic, super-paramagnetic or ferromagnetic material or any other material that responds to a magnetic field.

The particles, or a group of several particles in a complex, may be functionalized with a receptor that has a specific affinity to bind to or interact with a clinically relevant analyte. The receptor may be inherent to the particle itself. For example, the particle itself may be a virus or a phage with an inherent affinity for certain analytes. Additionally or alternatively, the particles can be functionalized by covalently or otherwise attaching or associating a receptor that specifically binds or otherwise recognizes a particular clinically-relevant analyte. The functionalized receptor can be an antibody, peptide, nucleic acid, phage, bacteria, virus, or any other molecule with a defined affinity for a target analyte. Other compounds or molecules, such as fluorophores or autofluorescent or luminescent markers, which may assist in interrogating the particles in vivo, may also be attached to the particles.

The wearable device may further include one or more data collection systems for interrogating, in a noninvasive manner, the functionalized particles present in a lumen of the subsurface vasculature in the local area of the wearable device. In one example, the wearable device includes a detector for detecting a response signal that is transmitted from the portion of subsurface vasculature in response to the interrogating signal. While not necessary in all cases, the wearable device may also include a signal source for transmitting an interrogating signal that can penetrate the wearer's skin into the portion of subsurface vasculature and induce a response signal that is related to the binding of the functionalized particles to the target analyte. The interrogating signal can be any kind of signal that is benign to the wearer and results in a response signal that can be used to detect binding of the clinically-relevant analyte to the functionalized particles.

In addition, the data collected by the wearable device may be analyzed, either locally on or remotely from the wearable device, to detect the presence or absence of the clinically-relevant analyte. In some examples, the data may be analyzed to further determine a concentration of the clinically-relevant analyte based on the response signal detected by the detector and determine whether a medical condition is indicated based on at least the presence, absence and/or concentration of the clinically-relevant analyte. As one possible example for use of the data collected by the wearable device, the presence of an unstable arterial plaque that could potentially cause a heart attack or stroke is often associated with an increase in certain protein markers in the blood. A person who may be at risk for this medical condition may take particles that are functionalized to bind to such protein markers and may wear on his or her wrist a device that is configured to periodically (e.g., every hour) collect and interrogate the functionalized particles to determine the concentrations of the protein markers. If the device determines that the concentrations of the protein markers indicate an elevated risk of a heart attack or other life-threatening episode, the device may generate an alert through the user interface (e.g., an audible alarm) so that the person wearing the device can seek immediate medical attention.

The functionalized particles may be introduced into the blood stream, or other bodily fluid, by injection, ingestion, inhalation, transdermally, or in some other manner. In one example, the functionalized particles are delivered to the gastrointestinal (GI) tract by a swallowable delivery device such as a capsule. As used herein, “GI tract” refers to the esophagus (E), stomach (S), small intestine (SI), large intestine (LI) and anus, while “Intestinal tract” refers to the small and large intestine. Various embodiments of the invention can be configured and arranged for delivery of medication 100 into both the intestinal tract as well as the entire GI tract. The capsule includes an interior volume and can be fabricated from various biocompatible polymers known in the art. The capsule can be fabricated from various non-toxic materials including various biodegradable polymers. The capsule may also have an enteric or other coating for protecting the capsule from stomach acids while allowing for biodegradation in the small intestine so as to allow the device to deliver functionalized particles into the wall of the small intestine responsive to pH or other conditions in the small intestine.

The delivery device described herein may be used for the delivery of functionalized particles to the gastrointestinal tract of a mammal, such as a human, canine, bovine or porcine intestinal tract. Characteristics of the delivery device and functionalized particles may be tailored for the particular mammal(s) under study. For example, in embodiments of the device employing various biodegradable materials, the pH under which those materials will degrade may be selected based on the pH of the target portion of the gastrointestinal tract of the chosen mammal.

In one embodiment, the capsule includes an expandable member and a tissue penetrating member advanceable into the intestinal wall by expansion of the expandable member. The capsule includes an interior volume and at least one aperture through which the tissue penetrating member can be advanced into the intestinal wall. In some examples, the expandable member is provided as a balloon disposed within the capsule interior volume and coupled to the tissue penetrating member. The balloon can be attached to an interior wall of the capsule in a least a partially non-expanded state and can comprise various non-compliant polymers known in the art such as PET, polyethylene and polyimide. The balloon may be thin walled e.g., less than about 0.02 millimeters.

In some embodiments, expansion of the balloon occurs by filling of the balloon with a gas, which may be achieved by a chemical reaction resulting in the production of carbon dioxide or other gas. The balloon may include at least a first and a second portion or compartment which are separated by be a separation valve or other separation means. A liquid, such as water, can be disposed within the first compartment and at least one reactant disposed in the second compartment which can be liquid though typically is solid. The reactants may include at least two reactants for example, an acid such as citric acid and a base such as sodium bicarbonate, which can have about a 1:2 ratio. Other reactants including other acids, e.g., acetic acid and bases are also contemplated. When the valve or other separation means opens, the reactants mix in the liquid and produce a gas such as carbon dioxide which expands the balloon and advances the tissue penetrating member into the intestinal wall as will be explained more fully herein. In addition to advancing the tissue penetrating members into tissue, the device can also be configured to have the inflated balloon break or otherwise separate apart the capsule into one or more pieces for easier passage through the intestinal tract.

The separation valve can be configured to open in a number of ways and responsive to a number of conditions. For example, the separation valve may be configured to open by having one or more portions degrade in response to the higher pH or other conditions found within the small intestine so that upon degradation, the valve opens. Degradation of the valve allows for mixing of the contents of the first and second compartments and, thus, expansion of the balloon. The separation valve can be positioned on the outside of the capsule or within the capsule interior where it is exposed to intestinal fluids which enter through the at least one aperture or other opening in the capsule. At least a portion of the capsule surface, including the portion containing the at least one aperture, may be coated with a protective layer, such as an enteric coating which also degrades in response to pH or other conditions within the small intestine. Such coatings provide a protective seal over the at least one aperture so that digestive fluids do not enter the capsule interior and start to degrade the separation valve until the capsule has reached the small intestine. As an alternative or additional embodiment, the valve may also be configured to open in response to compressive forces applied by a peristaltic contraction within the small intestine, after a certain period of time has elapsed, or in response to external activation by the patient.

In addition to the release valve, the balloon or other expandable member can also include a deflation valve which serves to deflate the expandable member after inflation. The deflation valve can comprise biodegradable materials which are configured to degrade upon exposure to the fluids in the small intestine and/or liquid in one of the compartments of the balloon so as to create an opening or channel for escape of gas within balloon. One or more puncture elements can also be attached to the inside surface of the capsule wall such that when the balloon fully deflates it contacts and is punctured by the puncture element.

Additionally, selectable portions of the capsule can be fabricated from such biodegradable materials so as to allow the entire device to controllable degrade into smaller pieces, facilitating passage and excretion through the GI tract. In some embodiments, the capsule can include seams of biodegradable material which controllably degrade to produce capsule pieces of a selectable size and shape to facilitate passage through the GI tract. The seams can be pre-stressed, perforated or otherwise treated to accelerate degradation.

The tissue penetrating member(s) may comprise a hollow needle or other like structure, with a lumen or other compartment and a tissue penetrating end for penetrating a selectable depth into the intestinal wall. The lumen may be pre-loaded or filled with functionalized particles. At least one guide tube, within which the penetrating member(s) may be disposed, may also be provided. In some examples, the capsule includes multiple tissue penetrating members and they may have a number of arrangements. Each of the penetrating members can carry the same or different types of particles (i.e., particles functionalized with a different receptor). The former provides for larger amounts of delivery of a particular type of particle, the later allows delivery of particles targeted for two or more different blood analytes at about the same time. The multiple tissue penetrating members may be symmetrically distributed or placed in other patters around the perimeter of the capsule or on the surface of the expandable member so as to anchor the capsule into the intestinal wall during delivery of the particles.

The tissue penetrating member can be fabricated from a biodegradable polymer such as PGLA so as to degrade within the small intestine and provide a fail-safe mechanism for detaching from the intestinal wall should it become retained there. In such embodiments, the penetrating member may be fabricated from a mixture of particles and biodegradable polymer, so as to deliver the particles upon degradation of the biodegradable polymer by the interstitial fluids within the wall tissue. The penetrating member may also typically include one or more tissue retaining features such as a barb or hook to retain the penetrating member within the tissue of the intestinal wall after advancement. The retaining features can be arranged in various patterns to enhance tissue retention such as two or more barbs symmetrically distributed around the member shaft. In further embodiments, the tissue penetrating member may also be fabricated from a drug, therapeutic agent, contrast agent or other substance configured to release the functionalized particles upon its degradation and absorption into the body.

As an additional or alternative embodiment to the use of particle-carrying tissue penetrating members, various embodiments of the device can also include reservoirs of particles disposed in the capsule which are compressible by expansion of the balloon or other expandable member. The reservoirs contain the particles either in a dry form, or suspended in a liquid. In these and related embodiments, the reservoirs are fluidically coupled to advanceable hollow tissue penetrating members such that inflation of the balloon compresses the reservoirs so as to force the particle suspension through tissue penetrating member and into the intestinal wall. Multiple reservoirs are contemplated including two, three, four or more. In particular embodiments, two reservoirs can be coupled to a hollow tissue penetrating member with the reservoirs placed about 180 degrees apart with respect to the lengthwise axis of the penetrating member. Typically, the reservoirs will be fluidically coupled to the hollow penetrating member by means of a connector. Suitable connectors include a t-shaped connector having connectors on either of it lateral ends for the reservoirs a central connector for the hollow tissue penetrating member and a central lumen or channel going to all connectors. Other shapes and connector configurations are also contemplated.

In other example capsules, advancement of the one or more tissue penetrating members is achieved with an actuator having an expandable member, delivery member and a release. The delivery member is configured to advance the particles from the capsule through the tissue penetrating member lumen and into the intestinal wall. At least a portion of the delivery member may be advanceable within the tissue penetrating member lumen and may be coupled to a portion of the actuator or to the expandable member. The actuator is configured to advance the tissue penetrating member a selectable distance into the intestinal wall as well as advance the delivery member to deliver the particles and then withdraw the tissue penetrating member from the intestinal wall. In some embodiments, such as where the tissue penetrating member is biodegradable, the actuator is configured to leave the tissue penetrating member within the intestinal wall. In various embodiments, the actuator can comprise a preloaded spring mechanism which is configured to be released by the release. Suitable springs can include both coil (including conical shaped springs) and leaf springs with other spring structures also contemplated. In particular embodiments, the spring can be cone shaped to reduce the length of the spring in the compressed state even to the point where the compressed length of the spring is about the thickness of several coils (e.g., two or three) or only one coil.

Release of the actuator may be controlled by a release coupled to at least one of the actuator or a spring coupled to the actuator. In particular embodiments, the release is coupled to a spring positioned within the capsule so as to retain the spring in compressed state. Degradation of the release triggers the spring to actuate the actuation mechanism. In many embodiments, the release comprises a material configured to degrade upon exposure to chemical conditions in the small or large intestine such as pH or other particular chemical conditions. Biodegradation of the release from one or more conditions in the small intestine (or other location in the GI tract) can be achieved by selection of material properties, such as the amount of cross linking of those materials as well as the thickness and other dimensions. Suitable materials for the release can comprise biodegradable materials such as various enteric materials which are configured to degrade upon exposure to the higher pH or other condition in the small intestine. In particular embodiments, the release can comprise a film or plug that fits over or otherwise blocks the guide tube and retains the tissue penetrating member inside the guide tube and/or capsule. In other embodiments, the release can be shaped to function as a latch which holds the tissue penetrating element in place. In these and related embodiments, the release can be located on the exterior or the interior of the capsule. In the interior embodiments, the capsule and guide tubes are configured to allow for the ingress of intestinal fluids into the capsule interior to allow for the degradation of the release.

In some embodiments, the actuator can be actuated by means of a sensor, such as a pH, chemical or mechanical sensor which detects the presence of the capsule in the small intestine and sends a signal to the actuator (or to an electronic controller coupled to the actuator to actuate the mechanism). Additionally or alternatively, the user may externally activate the actuator to deliver the particles by means of RF, magnetic or other wireless signaling means known in the art. In these and related embodiments, the user can use a handheld device (e.g., a hand held RF device) which not only includes signaling means, but also means for informing the user when the device is in the small intestine or other location in the GI tract. The user may also externally activate the actuator at a selected time period after swallowing the capsule. The time period can be correlated to a typical transit time or range of transit times for food moving through the user's GI tract to a particular location in the tract such as the small intestine.

Another aspect of the invention provides methods for the delivery of particles into the walls of the GI tract using embodiments of the swallowable particle delivery devices. The types and amounts of the particular particles delivered can be titrated for the patient's weight, age or other parameters and for the type of blood analytes for which analysis is desired. In various method embodiments, embodiments of the swallowable particle delivery device can be used to deliver a plurality of functionalized particles for the detection and analysis of one or more blood analytes. In use, such embodiments allow a patient to forgo the necessity of having to take multiple separate doses of particles. Also, they can facilitate a regimen of two or more types of particles that are delivered and absorbed into the small intestine and thus, the blood stream at about the same time. Due to differences in their size, shape, materials and functionalized receptors, different types of particles can be absorbed through the intestinal wall at different rates. Embodiments of the invention address this issue by injecting the particles at about the same time. Further, the various embodiments of the swallowable delivery device provide a means for delivering particles to the bloodstream via the GI tract that might otherwise require injection due to chemical breakdown in the stomach.

It should be understood that the above embodiments, and other embodiments described herein, are provided for explanatory purposes, and are not intended to be limiting.

Further, the term “medical condition” as used herein should be understood broadly to include any disease, illness, disorder, injury, condition or impairment—e.g., physiologic, psychological, cardiac, vascular, orthopedic, visual, speech, or hearing—or any situation requiring medical attention.

### II. ILLUSTRATIVE FUNCTIONALIZED PARTICLES

Health-related information of a patient may be obtained by detecting the binding of a clinically-relevant analyte to functionalized particles, for example, microparticles or nanoparticles, introduced into the body. The particles can be functionalized by covalently or otherwise attaching or associating a bioreceptor designed to selectively bind or otherwise recognize a particular clinically-relevant analyte. For example, particles may be functionalized with a variety of bioreceptors, including antibodies, nucleic acids (DNA, siRNA), low molecular weight ligands (folic acid, thiamine, dimercaptosuccinic acid), peptides (RGD, LHRD, antigenic peptides, internalization peptides), proteins (BSA, transferrin, antibodies, lectins, cytokines, fibrinogen, thrombin), polysaccharides (hyaluronic acid, chitosan, dextran, oligosaccharides, heparin), polyunsaturated fatty acids (palmitic acid, phospholipids), plasmids. In other examples, the particle itself may have an inherent receptor or affinity for a target analyte. For example, the particle itself may be a virus or phage with an inherent affinity for certain analytes. As used herein, the term “functionalized particles” may refer to any type, shaped or sized particle (i.e., spheres, rods, flakes, nano- or micro-particles, etc.) having an attached, associated or inherent bioreceptor that has an affinity for a particular blood analyte, disposed thereon or in the vicinity thereof.

The clinically-relevant analyte could be any analyte that, when present in or absent from the blood, or present at a particular concentration or range of concentrations, may be indicative of a medical condition or indicative that a medical condition may be imminent. For example, the clinically-relevant analyte could be an enzyme, hormone, protein, or other molecule. In one relevant example, certain protein biomarkers are known to be predictive of an impending arterial plaque rupture. Such protein biomarkers are known to be present in the blood only directly leading up to and at the onset of an arterial plaque rupture. Plaques that rupture cause the formation of blood clots that can block blood flow or break off and travel to another part of the body. In either of these cases, if a clot blocks a blood vessel that feeds the heart, it causes a heart attack. If it blocks a blood vessel that feeds the brain, it causes a stroke. If blood supply to the arms or legs is reduced or blocked, it can cause difficulty walking and eventually gangrene. The presence of these protein biomarkers in the vasculature may be detected, and the medical condition (i.e., stroke, heart attack) prevented, by providing particles functionalized with a bioreceptor that will selectively bind to this target analyte.

The particles may be made of biodegradable or non-biodegradable materials. For example, the particles may be made of polystyrene. Non-biodegradable particles may be provided with a removal means to prevent harmful buildup in the body. Generally, the particles may be designed to have a long half-life so that they remain in the vasculature or body fluids over several measurement periods. Depending on the lifetime of the particles, however, the user of the wearable device may periodically introduce new batches of functionalized particles into the vasculature or body fluids.

Bioreceptors can be used in diagnostic procedures, or even in therapy to destroy a specific target, such as antitumor therapy or targeted chemotherapy. The particles may be designed to remove from the body or destroy the target analyte once bound to the bioreceptor. Additional functional groups may be added to the particles to signal that the particles can be removed from the body through the kidneys, for example, once bound to the target analyte.

Further, the particles may be designed to either releasably or irreversibly bind to the target analyte. For example, if it is desired for the particles to participate in destruction or removal of the target analyte from the body, as described above, the particles may be designed to irreversibly bind to the target analyte. In other examples, the particles may be designed to release the target analyte after measurement has been made, either automatically or in response to an external or internal stimulus.

Those of skill in the art will understand the term “particle” in its broadest sense and that it may take the form of any fabricated material, a molecule, cryptophan, a virus, a phage, etc. Further, a particle may be of any shape, for example, spheres, rods, non-symmetrical shapes, etc. The particles can have a diameter that is less than about 20 micrometers. In some embodiments, the particles have a diameter on the order of about 10 nm to 1 μm. In further embodiments, small particles on the order of 10-100 nm in diameter may be assembled to form a larger “clusters” or “assemblies on the order of 1-10 micrometers. In this arrangement, the assemblies would provide the signal strength of a larger particle, but would be deformable, thereby preventing blockages in smaller vessels and capillaries.

Binding of the functionalized particles to a target analyte may be detected with or without a stimulating signal input. The term “binding” is understood in its broadest sense to include any detectable interaction between the receptor and the target analyte. For example, some particles may be functionalized with compounds or molecules, such as fluorophores or autofluorescent, luminescent or chemiluminescent markers, which generate a responsive signal when the particles bind to the target analyte without the input of a stimulus. In other examples, the functionalized particles may produce a different responsive signal in their bound versus unbound state in response to an external stimulus, such as an electromagnetic, acoustic, optical, or mechanical energy.

Further, the particles may be formed from a paramagnetic or ferromagnetic material or be functionalized with a magnetic moiety. The magnetic properties of the particles can be exploited in magnetic resonance detection schemes to enhance detection sensitivity. In another example, an external magnet may be used to locally collect the particles in an area of subsurface vasculature during a measurement period. Such collection may not only increase the differential velocity between particles and analytes, hence surveying a much larger volume per unit time, but may also enhance the signal for subsequent detection.

### III. EXAMPLE SWALLOWABLE DEVICES

One or more embodiments of the devices described herein can be used for the delivery of functionalized particles to the body for the identification and measurement of blood analytes to assess physiological parameters, which may indicate certain health-related conditions. The bioreceptors associated with or otherwise functionalized with the particles may include molecules, compounds or other substances that may be ill-suited for traditional oral delivery because they are susceptible to digestion, degradation or break-down by the digestive fluids in the stomach and/or the lumen of the small intestine. However, rather than being limited to delivering these sensitive functionalized particles via injection and/or IV infusion, they may be taken orally through use of the device. Embodiments of the delivery device allow functionalized particles to be delivered into the wall of the small intestine (or other targeted delivery site) and subsequently absorbed into the blood stream with minimal or no loss of activity of the functionalized receptor, e.g., in the case of an antibody, minimal or no loss in affinity and/or specificity to a target analyte; in the case of any polypeptide, minimal or no loss in affinity and/or specificity to a target analyte; etc. For receptors that would otherwise be partially degraded or poorly absorbed in the GI tract, the amount or dose of functionalized particles to achieve accurate identification and measurement of the target blood analyte can be less than the amount required should the particles have been delivered by conventional oral delivery (e.g., as a drinkable suspension of particles, or as a swallowable pill that is digested in the stomach and absorbed through the wall of the small intestine). Because the particles are delivered directly into the wall of the small intestine (or other lumen in the intestinal tract, e.g., large intestine, stomach, etc.), embodiments of the device described herein can provide protection to the sensitive functionalized particles, allowing for little or no degradation of the particles or their receptors by acid and other digestive fluids in the stomach.

Further, embodiments of the device provide an advantage of allowing for the delivery of multiple types of functionalized particles in a single dose or capsule. As described above, particles functionalized with different receptors may be used to identify and measure different blood analytes, each type of particles providing information about a different physiological parameter or an indication of a different aspect of the health state of the patient. In use, such embodiments allow a patient to forgo the necessity of having to take separate doses of particles, each specific to a particular target analyte. Also, the delivery device can enable a combination of functionalized particles and another agent, such as a contrast agent, fluorophore, enzyme, reactant, etc., to be delivered and absorbed into the small intestine and thus, the blood stream, at about the same time. Such timing may be necessary for the additional agent to provide some assistance or benefit to the action of the functionalized particles. Additionally, eliminating the need to take multiple doses of functionalized particles may be beneficial to patient compliance and timing.

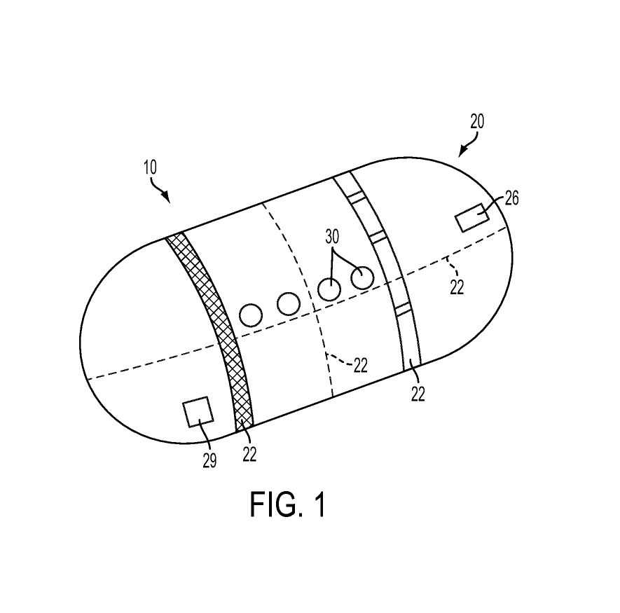

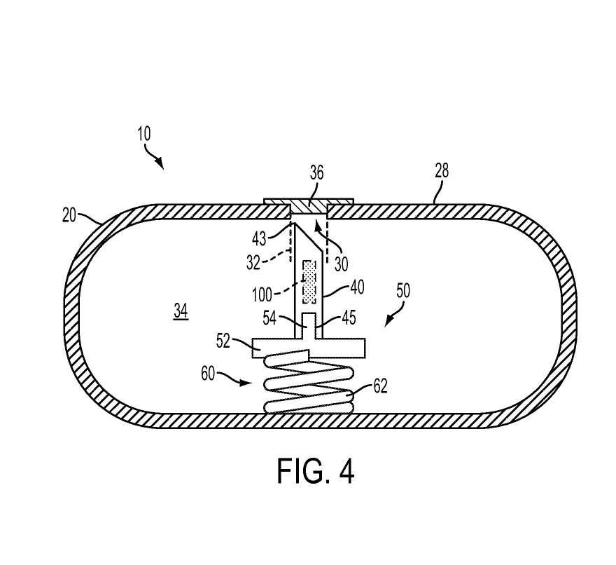

Referring now to the Figures, embodiments of a device 10 for the delivery of functionalized particles 100 to the intestinal tract are shown. As shown in FIGS. 1 and 4, in one embodiment, the device 10 may comprise a capsule 20 sized to be swallowed and pass through the intestinal tract, one or more tissue penetrating members 40, and actuator 50. Generally, capsule 20 may be provided in many sizes, depending on the target delivery site, the age, height, weight and gender of the patient, and the amount of functionalized particles intended to be delivered with the device 10. Capsule lengths can be in the range of 1 to 5 cm and diameters in the range of 0.25 to 1.5 cm, with other dimensions contemplated. The capsule 20 may be of any shape, including those known in the art, such as pill or tablet shaped.

One or more portions of capsule 20 can be fabricated from various biocompatible polymers known in the art, including various biodegradable polymers which in a preferred embodiment can comprise PGLA (polylactic-co-glycolic acid). Other suitable biodegradable materials include various enteric materials described herein as well as lactide, glycolide, lactic acid, glycolic acid, para-dioxanone, caprolactone, trimethylene carbonate, caprolactone, blends and copolymers thereof. Use of biodegradable materials for capsule 20, including biodegradable enteric materials allows the capsule to degrade in whole or part to facilitate passage through the GI system after delivery of the functionalized particles.

In some embodiments, the capsule 20 can include one or more seams 22, configured to segment the capsule 20 into two or more pieces. In some examples, the seams 22 may be pre-stressed, scored, or perforated regions configured to cause the capsule material to physically break, tear, rip or otherwise fail in those regions. In other examples, the seams 22 may be made of biodegradable material and can also include pores or gaps for ingress of fluids into the seam to accelerate biodegradation. The seams 22 may also be made from a material designed to biodegrade faster than the material chosen for the remainder of the capsule 20. In still other embodiments, seams 22 can be constructed of materials and/or have a structure which is readily degraded by absorption of ultrasound energy, e.g. high frequency ultrasound (HIFU), allowing the capsule to be degraded into smaller pieces using externally or endoscopically (or other minimally invasive method) administered ultrasound. Seams 22 can be attached to capsule body 20 using various joining methods known in the polymer arts such as molding, hot melt junctions, etc. Capsule 20 can also be fabricated from two or more separate joinable pieces that can be adhered together or, alternatively, joined by a mechanical fit such as a snap or press fit.

The capsule 20 can also include a marker 26 designed to assist in locating the capsule as it travels through the GI tract. Marker 26 may be fabricated from certain radio-opaque or echogenic materials for location of the device using fluoroscopy, ultrasound or other medical imaging modality. Use of a marker 26 may also allow for the determination of transit times of the device 10 through the GI tract.

One or more tissue penetrating members 40 are provided, as shown generally in FIG. 4, in the interior 34 of the capsule. In some embodiments, tissue penetrating members 40 are positioned within or aligned with guides 32 which may serve to guide members 40 through the one or more apertures 30 in the capsule wall 28 and into tissue, such as the wall of the small intestine or other portion of the GI tract.

A cap 36 may be provided to cover aperture 30 to protect the capsule interior 34 and its contents while the capsule 20 travels through the stomach and GI tract on its way to the target delivery site. As will be described further below, cap 36 fits over or otherwise blocks guide tubes 30 and may act to retain the tissue penetrating member 40 inside the guide tube 30. Cap 36 may be fabricated from a biodegradable material, chosen to degrade once the capsule reaches a certain region of the GI tract.

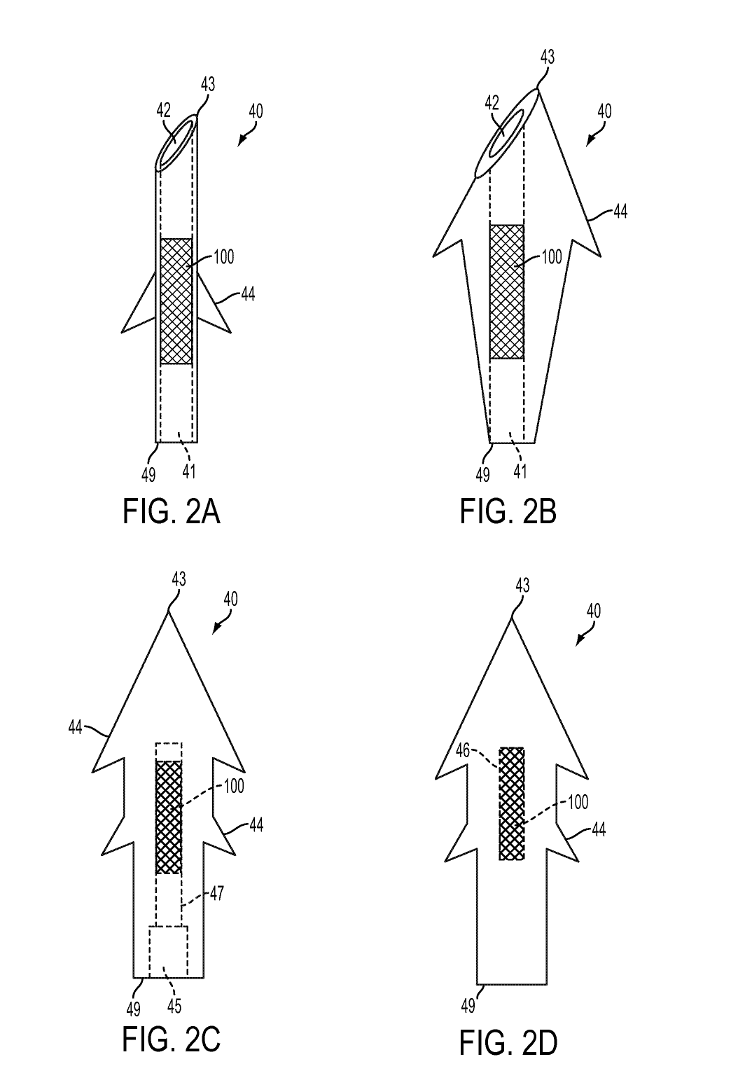

Turning to FIGS. 2A-2D, tissue penetrating members 40 may comprise a lumen 41, having an opening 42, and a tissue penetrating end 43, which may be pointed so as to readily penetrate tissue of the intestinal wall. In further examples, rather than having a lumen through which functionalized particles 100 may be delivered, tissue penetrating member 40 may instead have an internal compartment 46 in which a plurality of functionalized particles, or a preparation containing them, may be housed for delivery as shown in FIG. 2D. Tissue penetrating member may, in such examples, be fabricated from a biodegradable material so as to release functionalized particles 100 from compartment 46 upon degrading in the intestinal wall.

Tissue penetrating member may be designed to enhance the retention of tissue penetrating member 40 in the intestinal wall. In some examples, one or more retaining elements 44, such as a barb or hook, may be provided along the length of tissue penetrating member 40 to retain the penetrating member within the intestinal wall after deployment. Retaining elements 43 can be arranged in various patterns, longitudinally and/or radially, to enhance tissue retention. For example, as shown in FIG. 2C, two or more barbs may be distributed around and along member 40. In some embodiments, such as is shown in FIG. 2B, tissue penetrating member 40 may have a generally tapered shape. Peristaltic contractions from the intestinal tract acting on the tapered body may act to force or squeeze the member 40 farther into the intestinal wall.

Tissue penetrating member 40 can be fabricated from any biocompatible materials known in the art having the desired structural properties. In some examples, tissue penetrating members may be fabricated from one or more biodegradable polymers so as to degrade after delivery of functionalized particles 100. Such biodegradation can, as described above, act to release particles internally housed in the member 40, and to allow member 40 to be broken down and cleared from the body. Additionally, tissue penetrating members 40 may be fabricated from one or more other agents, such as medicinal, therapeutic or imaging contrast agents, which may provide some therapeutic or imaging enhancement to facilitate use of the functionalized particles 100. In some cases, the functionalized particles may be carried by the tissue penetrating member 40 by mixing them in with a biodegradable material, such as PGLA, cellulose or maltose, to form tissue penetrating member 40. Once delivered to the intestinal wall, the penetrating member 40 is degraded by the interstitial fluids within the tissue, thereby releasing the particles making up, in part, the member itself. Tissue penetrating member 40 can be fabricated using one or more polymer and pharmaceutical fabrication techniques known in the art, with particular attention paid to preventing any substantial thermal or chemical degradation of the functionalized particles.

The functionalized particles 100 may be delivered to the intestinal wall in a variety of ways. In general, the one or more tissue penetrating members 40 will be advanced into the intestinal wall via an actuator 50 and the functionalized particles will be delivered to the tissue via the one or more tissue penetrating members 40. The functionalized particles 100 themselves may be delivered on their own, in dry form, or in a preparation with another substance. For example, functionalized particles 100 may be combined with a pharmaceutically acceptable liquid to form a suspension preparation. Functionalized particles 100 may also be combined with any number of pharmaceutically acceptable gels, solids or powders to form solid or semi-solid preparations that may be designed to retain a particular shape, such as a pellet. Further, as described above, the preparation containing functionalized particles 100 may also include any number of other pharmaceutically acceptable excipients or substances, such as drugs, or therapeutic or imaging agents.

As shown in FIGS. 2A and 2B, the functionalized particles may be pre-packed within the lumen 41 of tissue penetrating members 40. In other examples, as shown in FIGS. 2C and 2D, delivery of the functionalized particles 100 can be achieved through degradation of the tissue penetrating member itself. Tissue penetrating member can include a passage 47 into which functionalized particles may be introduced and housed for delivery, as shown in FIG. 2C. Alternatively, the tissue penetrating member 40 can include an integral internal compartment 46 containing functionalized particles 100 into which functionalized particles 100 are introduced during manufacture of member 40. As described above, the functionalized particles 100 may also be mixed with a biodegradable polymer and used to fabricate the body of the tissue penetrating member 40 itself. It is also contemplated that functionalized particles can be contained at another location within an interior 34 of capsule 20.

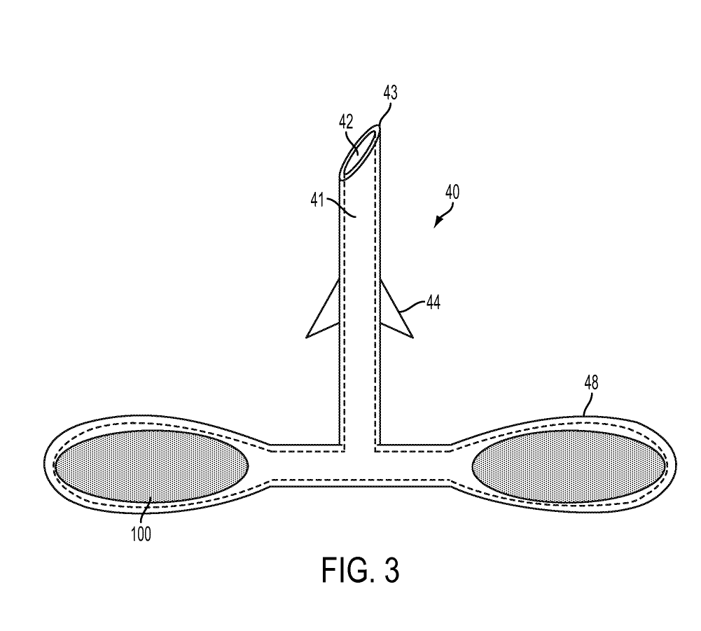

Tissue penetrating members 40 may also be fluidically connected to one or more reservoirs 48 containing functionalized particles. In one example shown in FIG. 3, tissue penetrating member 40 is connected to two reservoirs 48. The reservoir 48 may be made of a compressible material, whereby compression thereof acts to force functionalized particles 100 contained therein into the lumen 40 and into the issue via opening 42. The reservoirs 48 can contain the functionalized particles 100, or a preparation containing them, in a dry or suspended form.

The device 10 can be configured for delivery of a single or of multiple types of functionalized particles 100. If multiple tissue penetrating members 40 are provided, each may be used to deliver a different type of functionalized particle. Similarly, different types of particles can be contained within separate compartments or reservoirs 48 within capsule 20.

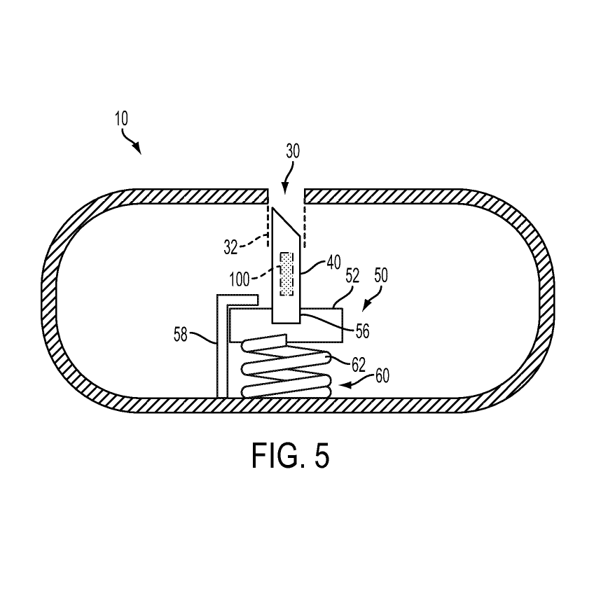

Device 10 also includes an actuator 50 coupled, either directly or indirectly, to the at least one tissue penetrating member 40. The actuator 50 is configured to advance the functionalized particles 100 from within the capsule into a wall of a lumen of the gastrointestinal tract via the one or more tissue penetrating members 40. In some embodiments, the actuator 50 may also be configured to withdraw the tissue penetrating member 40 from the intestinal wall. The actuator 50 may include an expandable member 60, which can comprise a variety of expandable devices shaped and sized to fit within capsule 20. In some examples, expandable member comprises a spring 62, as shown in FIGS. 4 and 5. Spring 62 can include both coil (including conical shaped springs) and leaf springs with other spring structures also contemplated. In other examples, expandable member comprises an expandable balloon 64. Other suitable expandable members include various shape memory devices, and/or chemically expandable polymer devices having an expanded shape and size corresponding to the interior volume 34 of the capsule 20.

Generally, actuator 50 has at least a first, or retracted, configuration and a second, or deployed, configuration. In the first configuration, actuator 50 is configured to retain the functionalized particles within the capsule. The actuator 50 is configured to transition from the first configuration to the second configuration, thereby advancing the plurality of functionalized particles from the capsule into an intestinal wall. Transitioning from the first to the second configuration may be achieved by expansion of expandable member 60.

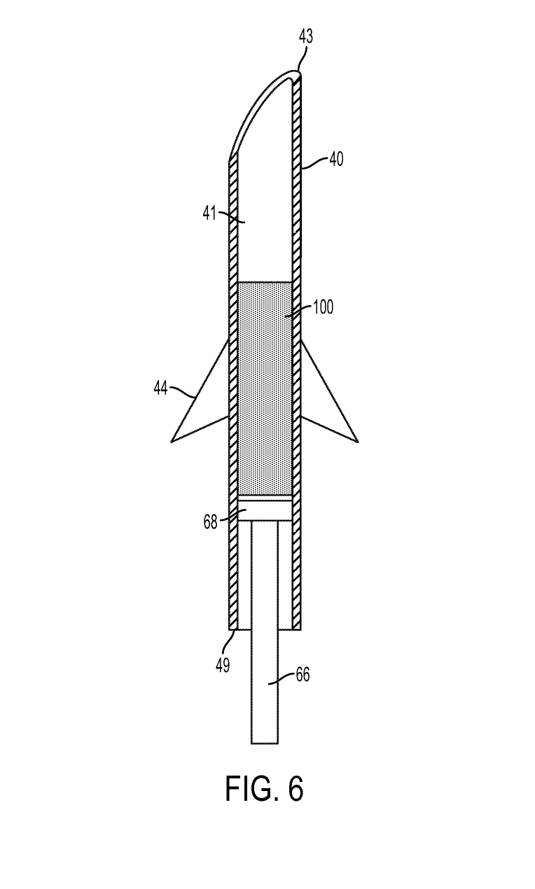

Actuator 50 may also include a connector 52, on or in which tissue penetrating members 40 may be placed, which may serve to stabilize members 40 and couple them to actuator 50. Connector 52 may include a key 54 for mating with a notch 45 on tissue penetrating member 40. Alternatively, connector 52 may include an inset 56 for tissue penetrating member 40 to fit therein. A release 58 designed to releasably maintain the actuator in the first configuration may be directly or indirectly coupled to one or more of the actuator 50, expandable member 60 or tissue penetrating member 40. In some embodiments, the release can mechanically block the guide tube and retain the tissue penetrating member inside the guide tube and/or capsule. For example, cap 36 as shown in FIG. 4 may physically act on tissue penetrating member 40 to retain spring 62 in a compressed state. In other embodiments, the release element can be shaped to function as a latch which holds the tissue penetrating member 40 in place or the expandable member 60 in a contracted state, as shown in FIGS. 5 and 6a.

In a further example, actuator 50 further includes a plunger 66, at least partially slidably received within the tissue penetrating member lumen 41, as shown in FIG. 6. Plunger may be connected directly to the expandable member 60 or to the connector 52 and is configured to advance the particles 100 through the tissue penetrating member lumen and into the intestinal wall. Plunger 66 may also include a head 68, which may be sized to approximately match the diameter of lumen 41.

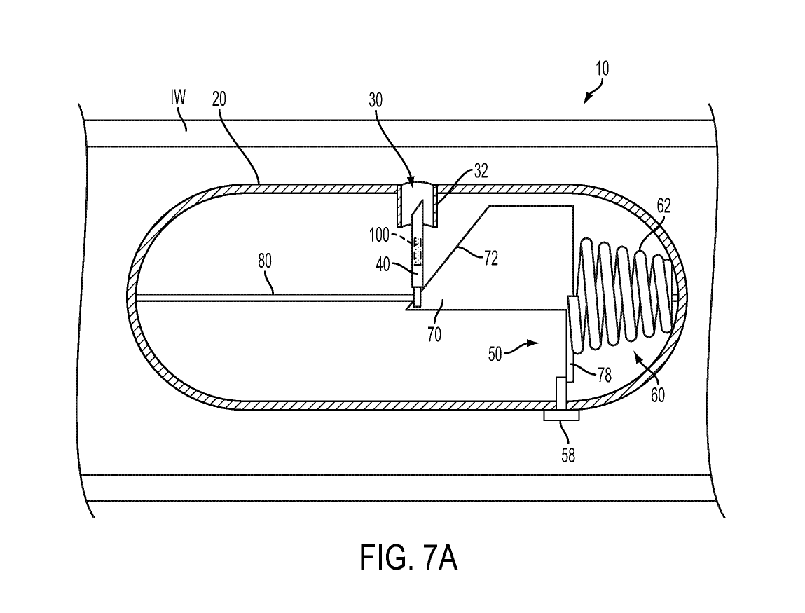

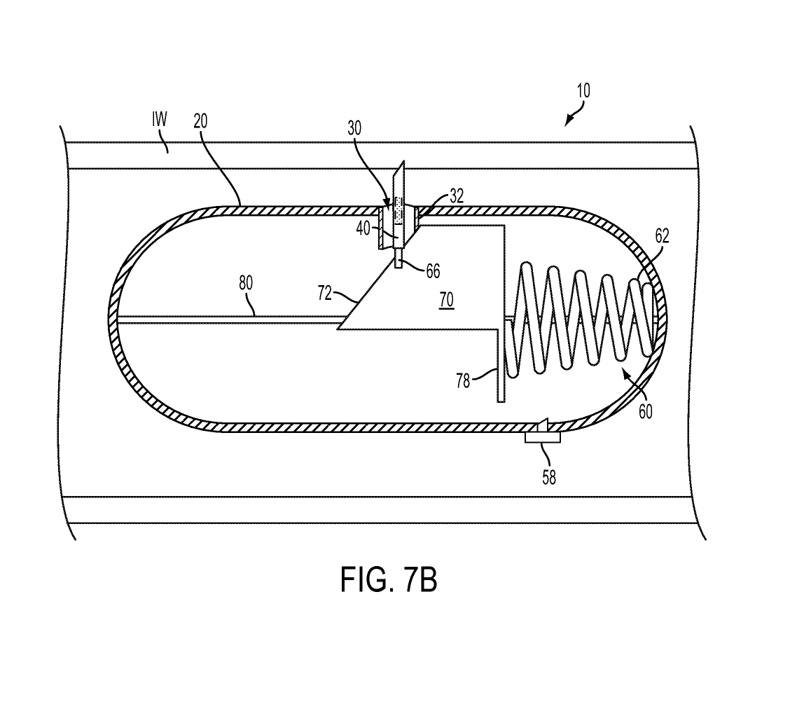

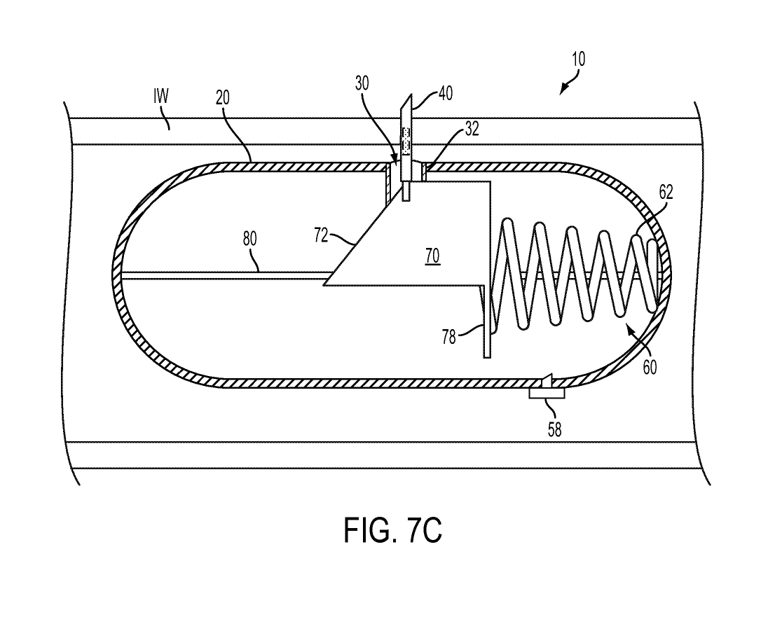

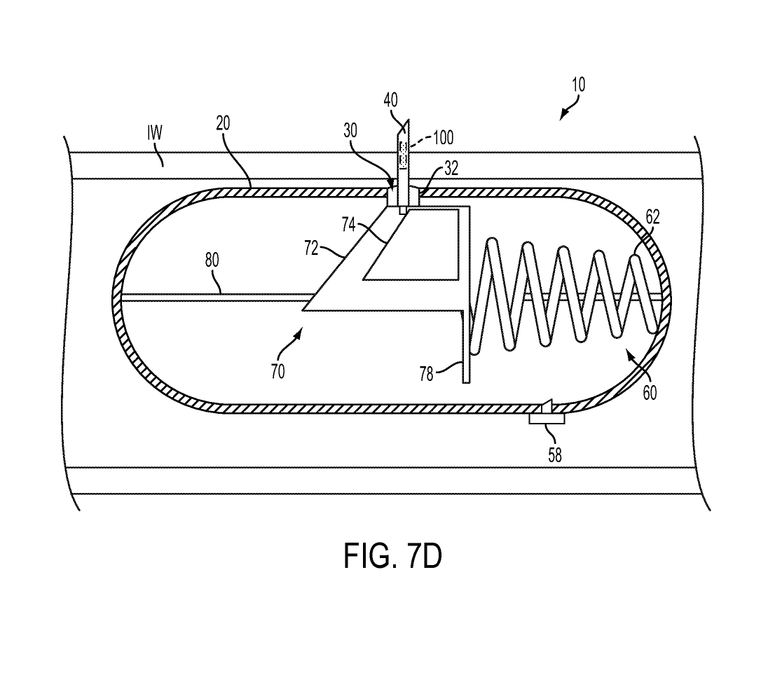

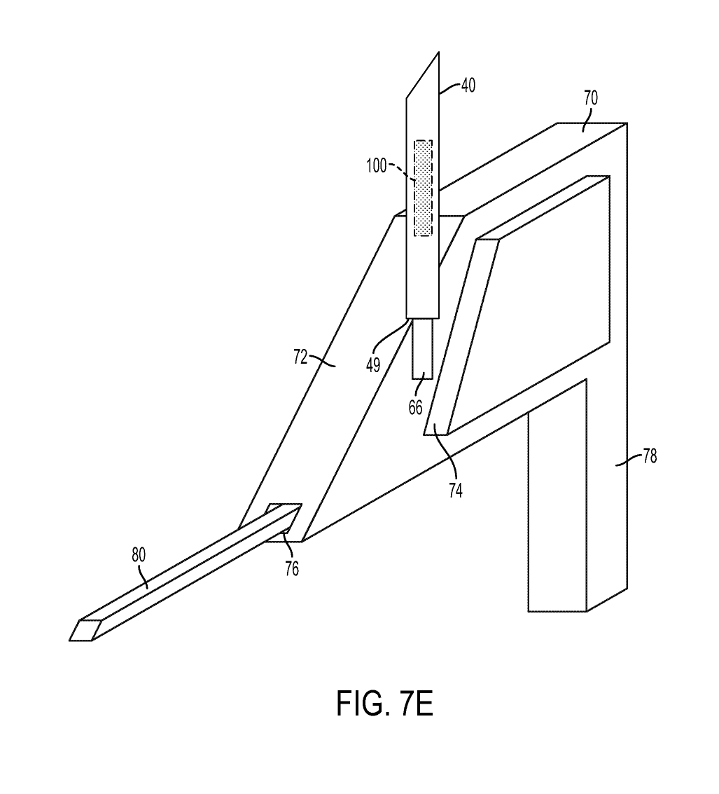

Actuator 50 may be configured such that expandable member 60 directly or indirectly advances tissue penetrating members 40 through apertures 30 and into the intestinal wall. In the embodiments shown in FIGS. 4 and 5, expansion of spring 62 directly advances expandable member 40 out through aperture 30 in the direction of expansion. As will be described further below, actuation of the expandable member can be inhibited by a release until the device 10 reaches the target delivery site. In another embodiment, shown in FIGS. 7A-7E, actuator 50 comprises an expandable member 60, in the form of spring 62 and a ramp 70. Ramp 70 may include a first incline 72 for engaging a portion of tissue penetrating member 40, such as the lower edge 49 of member 40. In some examples, ramp 70 may include a second incline 74 for engaging plunger 66, as shown in FIG. 7E.

Ramp 70 is pushed by the expandable member 60 (spring 62) along a rod 80, which is slidably received in track 76 passing through ramp 70. Both rod 80 and track 76 may be non-circular in shape so as to prevent ramp 70 from rotating around rod 80. As ramp 70 is advanced along rod 80, lower edge 49 of tissue penetrating member 40 engages first incline 72 (FIG. 7B). Tissue penetrating member 40 may be held in place longitudinally by, in some embodiments, guide 32 such that longitudinal advancement of ramp 70 translates into direct upward movement of tissue penetrating member 40. First incline 72 is sized and shaped to advance tissue penetrating member through aperture 30 and into intestinal wall IW (FIG. 7C). Second incline 74, configured to engage plunger 66, may be offset from first incline 72 in the direction of travel, as shown in FIG. 7D, such that plunger 66 is not slidably advanced into lumen 41 of tissue penetrating member until member 40 has engaged the intestinal wall IW. Ramp 70 may also include a reverse incline (not shown) for engaging lower edge 49 and withdrawing tissue penetrating member 40 from the intestinal wall after member 40 has travelled up first incline 72. One or more components of actuator 50 (as well as other components of device 10) including ramp 70 and rod 80 can be fabricated using various MEMS-based methods known in the art so as to allow for selected amounts of miniaturization to fit within capsule 20. Also as is described herein, they can be formed from various biodegradable materials known in the art.

As generally described above, release 58 is configured to retain actuator 60 in a first, or undeployed, configuration. Multiple releases 58 may also be provided within the capsule to trigger one or more actuators 60 and not necessarily in response to the same condition. In some examples, release 58 can comprise a film or plug that fits over aperture 30 or otherwise blocks guide tube 32 and retains the tissue penetrating member 40 inside the guide tube 32. Cap 36 shown in FIG. 4 may serve this purpose. In other examples, release 58 may be configured to directly retain expandable member 60 in a compressed state. For example, as shown in FIG. 5, release 58, in the form of a latch, compresses spring 62. In the embodiment shown in FIGS. 7A-7E, release 58 engages stop 78 on ramp 70, preventing spring 62 from expanding. In these and other embodiments, the release 58 can be located on the exterior or the interior of capsule 20. In the latter case, capsule 20 and/or guide tubes 32 can be configured to allow for the ingress of intestinal fluids into the capsule interior to allow for the degradation of the release.

Generally, release 58 is configured to activate actuator 60 once the device 10 has arrived at the target delivery site in the intestinal tract. Release 58 may be triggered in a number of manners, including degradation of the release 58 itself. In some examples, release 58 may be configured to degrade in response to a chemical condition in the gastrointestinal tract, such that degradation of the release causes the actuator to transition from the first configuration to the second configuration. For example, release 58 may be fabricated from a material configured to degrade upon exposure to chemical conditions in the small or large intestine such as pH. Release 58 may be configured to degrade upon exposure to a selected pH in the small intestine, e.g., 7.0, 7.1, 7.2, 7.3, 7.4, 7.5, 7.6 8.0 or greater. In some examples, release 58 is configured to degrade in a pH range from 7.0 to 7.5.

Release 58 can also be configured to degrade in response to other conditions in the small intestine (or other GI location). In particular embodiments, the release 58 can be configured to degrade in response to particular chemical conditions in the fluids in the small intestine such as those which occur after ingestion of a meal (e.g., a meal containing fats, starches or proteins). In this way, the release of functionalized particles 100 can be substantially synchronized or otherwise timed with the digestion of a meal.

Suitable enteric or biodegradable materials for the release include, but are not limited to, the following: cellulose acetate phthalate, cellulose acetate trimellitate, hydroxypropyl methylcellulose phthalate, polyvinyl acetate phthalate, carboxymethylethylcellulose, copolymerized methacrylic acid/methacrylic acid methyl esters as well as other enteric materials known in the art. The selected enteric materials can be copolymerized or otherwise combined with one or more other polymers to obtain a number of other particular material properties in addition to biodegradation. Such properties can include without limitation stiffness, strength, flexibility and hardness.

Additionally or alternatively, release 58 may also be provided as or with a sensor, such as a pH sensor or other chemical sensor which detects the presence of the capsule 20 in the small intestine, thereby triggering release of the actuator 50. Embodiments of a pH sensor can comprise an electrode or a mechanically-based sensor such as a polymer which shrinks or expands upon exposure to a selected pH or other chemical conditions in the small intestine. In other examples, release 58 may comprise an expandable/contractible sensor, configured to release actuator 50 using the mechanical motion from the expansion or contraction of the sensor. Release 58 may also be provided with or as a pressure/force sensor such as a strain gauge for detecting the number of peristaltic contractions that capsule 20 is being subject to within a particular location in the intestinal tract and may be configured to release actuator 50 once it has reached the desired delivery site.

In the embodiment shown in FIGS. 7A-7E, once the capsule 20 reaches the small intestine, the release 58 is degraded by the basic pH in the small intestine (or other chemical or physical condition unique to the small intestine) so as to release expandable member 60, provided as a spring 62, actuate the actuator 50 and deliver functionalized particles 100 into the intestinal wall IW. For embodiments including a hollow tissue penetrating member 40, delivery may be effectuated by using the actuator 50 to advance the penetrating member 40 a selected distance into the mucosa of the intestinal wall IW, and then injecting the functionalized particles through the lumen opening 42 by advancement of the plunger 66.

In some examples, actuator 50 may be configured to withdraw tissue penetrating members 40 back within the body of the capsule (e.g. by recoil), detaching from the intestinal wall. In other examples, after delivery to the intestinal wall, tissue penetrating member(s) 40 may be detached from the actuator 50 and retained in the tissue. For example, tissue penetrating member 40 may be configured to be detachably coupled (directly or indirectly) to the expandable member 60, such as a spring 62 or balloon 64 (as described below), so that after advancement of the tissue penetrating member 40 into the intestinal wall, the penetrating member detaches from the expandable member 60. Detachability can be implemented by a variety of means including: i) the configuration and strength of the joint between penetrating member 40 and actuator 50 or other intermediary component(s), such as connecter 52; 2) the configuration and placement of tissue retaining features 44 on penetrating member 40; and iii) the depth of penetration of tissue penetrating member 40 into the intestinal wall. Using one or more of these means, penetrating member 40 be configured to detach as a result of retraction of the expandable member 60 (where the retaining features 44 hold the penetrating member in tissue as expandable member detracts or otherwise pulls back away from the intestinal wall) and/or the forces exerted on capsule 20 by a peristaltic contraction of the small intestine.

After delivery, device 10 and its components degrade, at least in part, then pass through the intestinal tract including the large intestine and are ultimately excreted. Where the capsule 20 is tearable and/or has biodegradable seams 22 or other biodegradable portions, the capsule may break down into smaller pieces in the intestinal tract to facilitate passage through and excretion from the body. In particular embodiments, tissue penetrating members 40 can be biodegradable. Thus, should the member get stuck in the intestinal wall, it may biodegrade releasing the capsule 20.

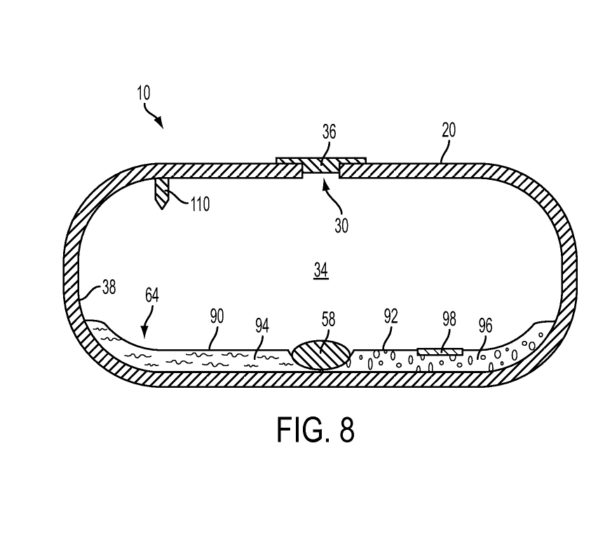

Turning now to FIG. 8, in other embodiments of device 10, expandable member 60 of actuator 50 may be provided as a balloon 64. Balloon 64 can be attached to an interior surface 38 of the capsule 20 in a non-expanded state. Means of attachment can include the use of various adhesive known in the medical device arts. The balloon can be packed inside capsule 20 in a furled or other compact configuration to conserve space within the interior portion of the capsule.

Balloon 64 can be fabricated from various polymers, including types of polyethylene (PE) which may correspond to low density PE(LDPE), linear low density PE (LLDPE), medium density PE (MDPE) and high density PE (HDPE) and other forms of polyethylene known in the art. The material may be cross-linked using polymer irradiation methods known in the art to control the inflated diameter and shape of the balloon by decreasing the compliance of the balloon material. Other suitable polymers can include PET (polyethylene teraphalate), silicone and polyurethane. Balloon 64 may also include various radio-opaque materials known in the art such as barium sulfate to allow a physician to ascertain the position and physical state of the balloon (e.g., un-inflated, inflated or punctured).

The balloon 64 may be fabricated using various balloon blowing methods known in art (e.g., mold blowing) to have a shape and size which corresponds approximately to the interior volume 34 of capsule 20. In some embodiments, the inflated size of the balloon can be configured to provide improved contact between the capsule/balloon surface and the intestinal wall so as to effectively deploy tissue penetrating members 40 and deliver functionalized particles 100. For example, the balloon can be sized, such that when inflated, it smooths the folds of the small intestine. In some embodiments, the inflated size of balloon 64 can be slightly larger than capsule 20 so as to cause the capsule to come apart or otherwise fail from the force of inflation. The walls of balloon 64 may have a thickness in the range of 0.1 to 0.002 mm. In some embodiments, the walls of the balloon 64 have a thickness in the range of 0.02 to 0.002 mm. In further embodiments, the walls of the balloons 64 may be provided with wall thicknesses of 0.013, 0.01, 0.007, 0.005, or 0.003 mm.

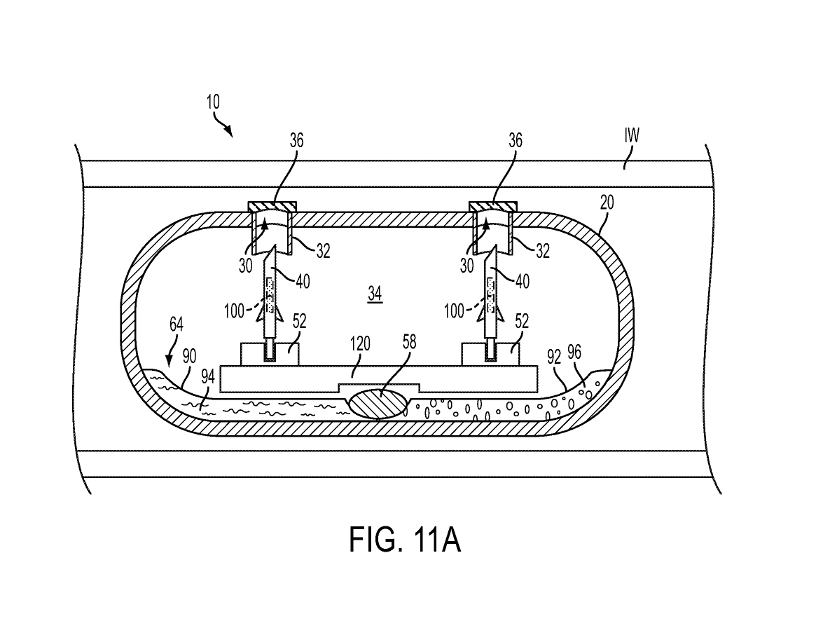

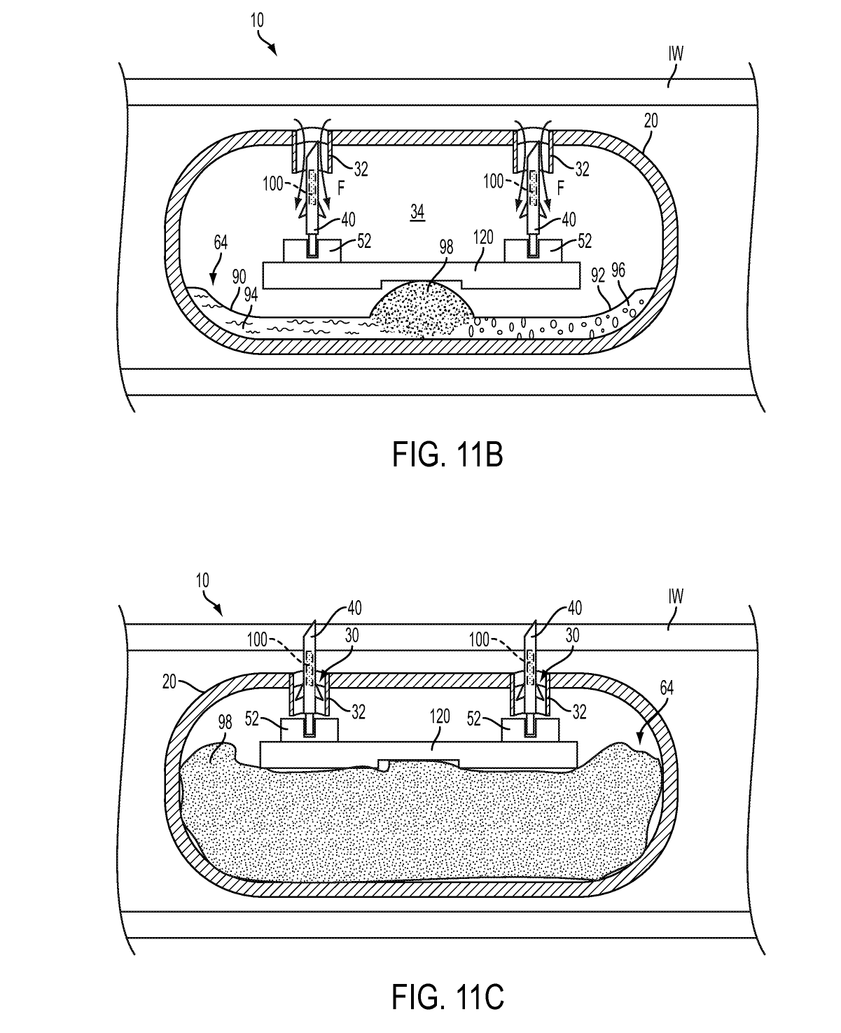

Balloon 64 may include at least first 90 and second 92 compartments which are separated by a release 58, which separates the contents of each compartment. First and second compartments 90, 92 each house a substance that, when mixed, will react to generate a gas that will expand balloon 64. A liquid 94, in some cases water, can be disposed within first compartment 90 and one or more reactants 96 disposed in second compartment 92. Reactants 96 may be solids or liquids. When release 58 is triggered (e.g., from degradation caused by fluids within the small intestine), liquid 94 enters second compartment 92 (or vice versa or both), the reactant(s) 96 mix with the liquid and produce a gas 98, such as carbon dioxide, which expands balloon 64 as is shown in the embodiments of FIGS. 11A-11C. Expansion of balloon 64 is configured to advance functionalized particles 100 into the intestinal wall IW, via the one or more tissue penetrating members 40.

Reactants 96 may include an acid such as citric acid and a base such as sodium bicarbonate. Additional numbers of reactants are also contemplated. For embodiments using citric acid and sodium bicarbonate, the ratios between the two reactants (citric acid to sodium hydroxide) can be in the range of 1:1 to 1:4, with a specific ratio of 1:2. Solid reactants, such as sodium bicarbonate, can be pre-dried (e.g., by vacuum drying) before being placed within balloon 64. Other reactants 96, including acetic acid are also contemplated. The amounts and selected combinations of particular reactants 96 can be chosen to produce particular pressures using known stoichiometric equations for the particular chemical reactions as well as the inflated volume of the balloon and the ideal gas law (e.g., PV=nRT).

Balloon 64 or other expandable member 60 may also include one or more deflation valves 98 which serve to deflate balloon 64 after inflation, as shown in FIG. 8. Deflation valve 98 can be fabricated from biodegradable materials which are configured to degrade upon exposure to the fluids in the small intestine and/or liquid in one of the compartments of the balloon so as to create an opening or channel for escape of gas within balloon. Multiple deflation valves 98 can be placed at various locations within balloon wall to provide an even higher degree of reliability in deflation. In general, deflation valve 98 may be fabricated from a degradable material designed to degrade more slowly than the release 58, allowing time for balloon 64 to fully inflate and deliver functionalized particles 100 to the intestinal wall before degrading and deflating the balloon. Additionally, as further backup for insured deflation of balloon 64, one or more puncture elements 110 can be attached to the inside surface 38 of the capsule wall such that when the balloon fully inflates, it contacts and is punctured by the puncture element. Other means for balloon deflation are also contemplated.

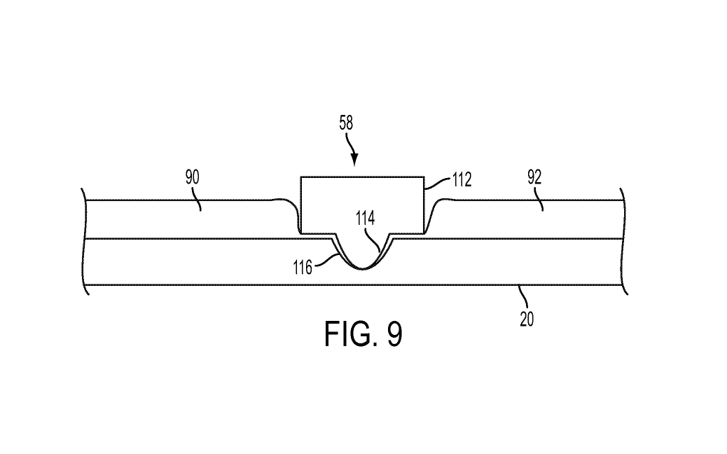

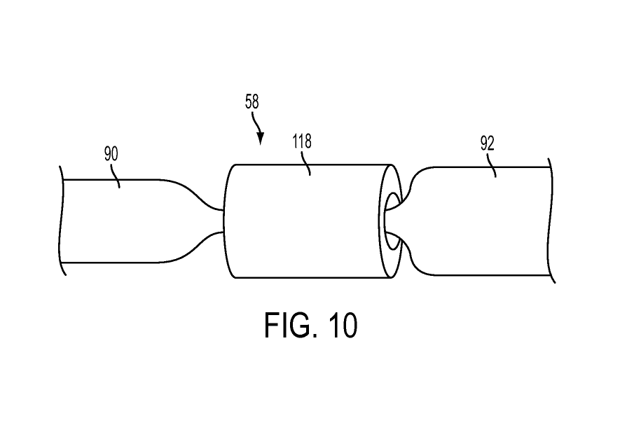

Release 58 may be provided in a number of structures and configurations, including, for example, a pinch valve 112 (FIG. 9) or collar 118 (FIG. 10). Still other structures are considered. In one embodiment, shown in FIG. 9, pinch valve 112 can include one or more protrusions 114 shaped to pinch balloon 64 into a depression 116 on the internal surface 38 of capsule 20. Multiple protrusions 114 may be used to create multiple seal points. According to another embodiment, shown in FIG. 10, the release 58 can comprise a collar 118 for constricting balloon 64 to maintain separation between the first and second compartments 90, 92.

Release 58, such as pinch valve 112 or collar 118, can be configured to open in a number of ways and responsive to a number of conditions within the GI tract. In some embodiments, release 58 will be configured to open by having one or more portions degrade in response to the higher pH or other conditions found within the small intestine. Accordingly, release 58 may be made from biodegradable material, thereby acting to seal first and second compartments 90, 92 and releasing them when upon degradation. Release 58 may also be configured to open in response to compressive forces applied by a peristaltic contraction within the small intestine. In still another approach, release 58 may be a time-release valve configured to open after a certain period of time after a trigger event, e.g., an activation step initiated by the patient. In a further embodiment, release may be provided as or with an expandable/contractible pH sensor, configured to expand or contract so as to open a channel between balloon compartments 90 and 92, in response to sensing a particular pH, particularly upon exposure to the pH conditions in the small intestine (e.g., a pH above 6.0, 6.5, 7.0, 7.1, 7.2, etc.).

Further, in some embodiments, at least a portion of the capsule exterior surface, including the portion containing the at least one aperture 26, may be covered with a protective layer or coating, such as an enteric coating which also degrades in response to pH or other conditions within the small intestine. At the very least, the coating may cover aperture 26, in the form of cap 36 for example, so that digestive fluids do not enter the capsule interior 34 and degrade the release 58 until the capsule has reached the small intestine.

Tissue penetrating member 40 can be directly or indirectly coupled to balloon 64. In some embodiments, tissue penetrating member 40 may be positioned in a connector 52, to stabilize the member 40 and hold it in the correct position. In further embodiments, tissue penetrating member 40 may be coupled to a platform 120. Platform 120 may comprise a rigid structure attached to the balloon surface on one side and attached to a connector 52 on the other, which releasably engages the penetrating member 40. Connector 52 may be an independent component, as shown in FIG. 11A, or may be formed integrally with platform 120. Both connectors 52 and platform 120 may be constructed from biodegradable materials such as PGLA, which can be cross linked and/or copolymerized to have increased rigidity to support the advancement of penetrating members 40 into tissue. Tissue penetrating members 40 can also be directly coupled to platform 120 without necessarily using a connector 52, for example by using a protrusions, indentations, or adhesives (not shown). Further, tissue penetrating members 40 may be directly coupled to the balloon 64 e.g., by an adhesive where the adhesive force is less than the necessary to pull penetrating member out of tissue once it is deployed into the intestinal wall. In these and related embodiments, the tissue penetrating members 40 may also be configured to rupture the balloon wall when they detach from the balloon and thus provide a means for balloon deflation.

Connector 52 can be configured such that tissue penetrating member 40 will detach therefrom in response to the force of balloon deflation and/or force applied to capsule 20 by peristaltic contraction. In some embodiments, platform 120 can have a larger horizontal surface area than the surface area of penetrating member 40 so as to function as a force concentration element. In use, platform 120 may function to increase the force per unit area applied to the penetrating member from expansion of balloon 64 or other expandable member. Other structures for loading tissue penetrating members 40 and coupling them to the expandable member 60 are contemplated.

The embodiments of FIGS. 11A-11C illustrate a sequence of degradation of the caps 36, ingress of intestinal or other fluid F into the capsule interior 34 and subsequent degradation of the release 58. In use, embodiments of device 10 employing a degradable cap 36 to cover the aperture 26 and a degradable release 58 provide a primary and secondary seal for assuring that balloon 64 does not prematurely expand and deploy its tissue penetrating members 40 until capsule 20 has reached the small intestine. Upon ingress of intestinal fluid F into the interior 34 of capsule 20, release 58 degrades, allowing liquid 94 disposed within first compartment 90 to mix with the one or more reactants 96 disposed in the second compartment 92 to form a gas 98 (FIG. 11B). As gas 98 is generated, balloon 64 expands filling the interior 34 of capsule 20 thereby forcing platform 120, and coupled tissue penetrating members 40 through guides 32 and out apertures 30. Expansion of balloon 64 forces tissue penetrating members 40 into the intestinal wall IW thereby delivering functionalized particles 100.

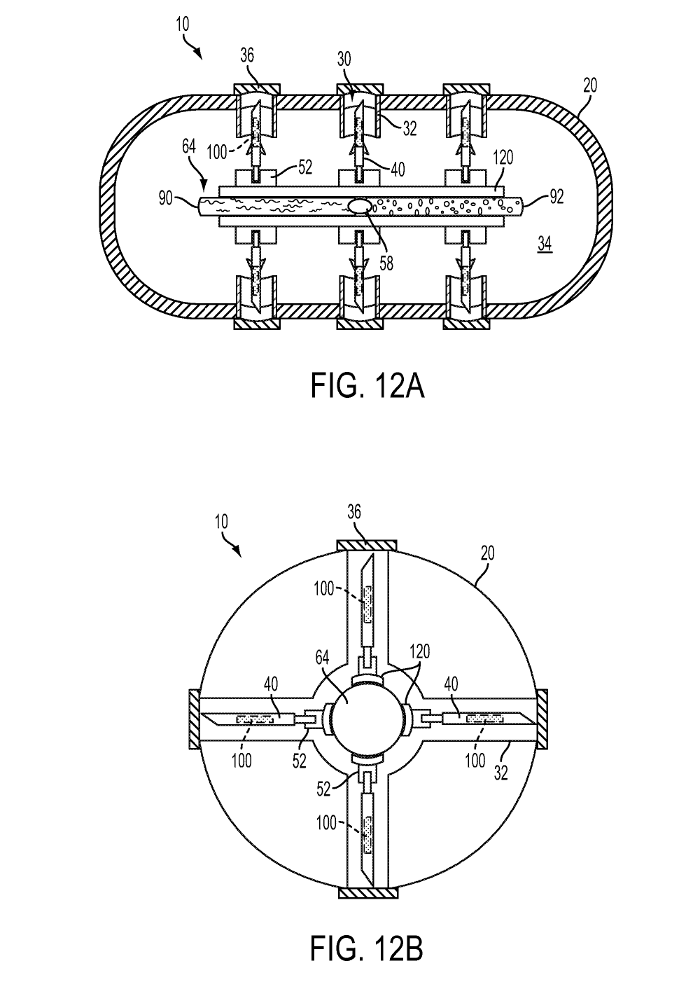

Tissue penetrating members 40 can be placed and distributed in a number of locations and patterns on the balloon surface. For example, as shown in FIG. 12A, platforms 120 can be placed on either side of balloon 64 to allow for bilateral deployment of multiple tissue penetrating members 40 into intestinal wall IW. In addition to delivering more functionalized particles 100 at once, bilateral deployment serves to anchor capsule 20 on both sides of the intestinal wall IW during deployment of penetrating members 40, thus reducing the likelihood of the capsule from being dislodged during deployment (e.g., due to peristaltic contraction). Multiple tissue penetrating members 40 may also be positioned radially around the expansion member 64, as shown in FIG. 12B, and along its length. Use of such a distributed delivery of functionalized particles 100 into the intestinal wall can also provide for faster absorption of the functionalized particles into the blood stream due to a more even distribution of the functionalized particles within the intestinal wall.

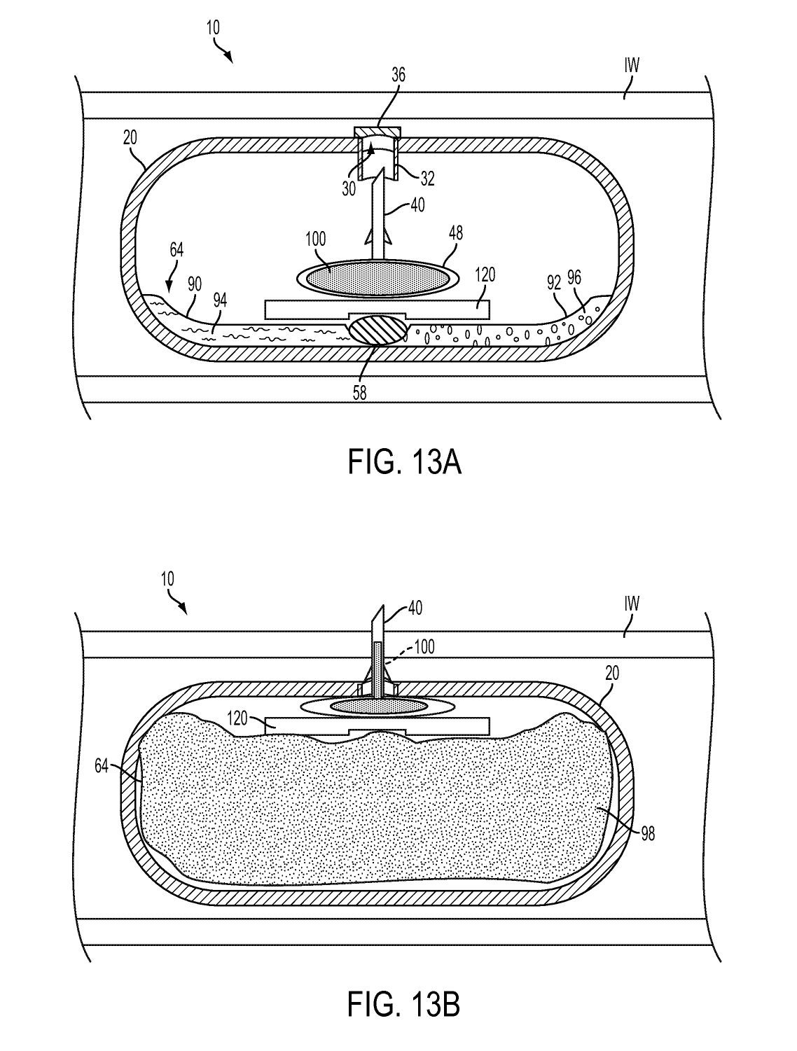

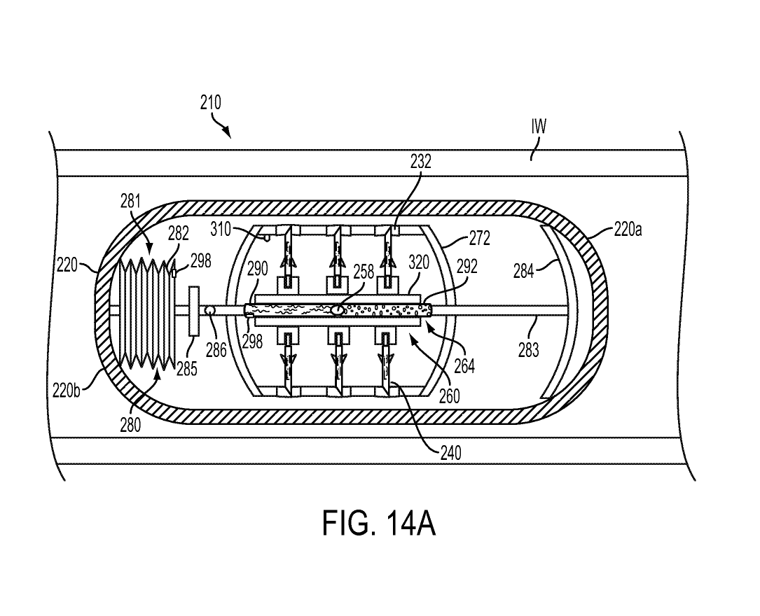

Turning to FIGS. 13A and 13B, tissue penetrating members 40 may also be coupled to one or more reservoirs 48 containing functionalized particles 100. The reservoir 48 may be fluidically coupled to tissue penetrating member 40 such that inflation of balloon 64, or expansion of some other expandable member 60, compresses the reservoirs 48 so as to force the functionalized particles, or a preparation thereof, through the lumen of tissue penetrating member 40 and into the intestinal wall IW, as shown in FIG. 13B.

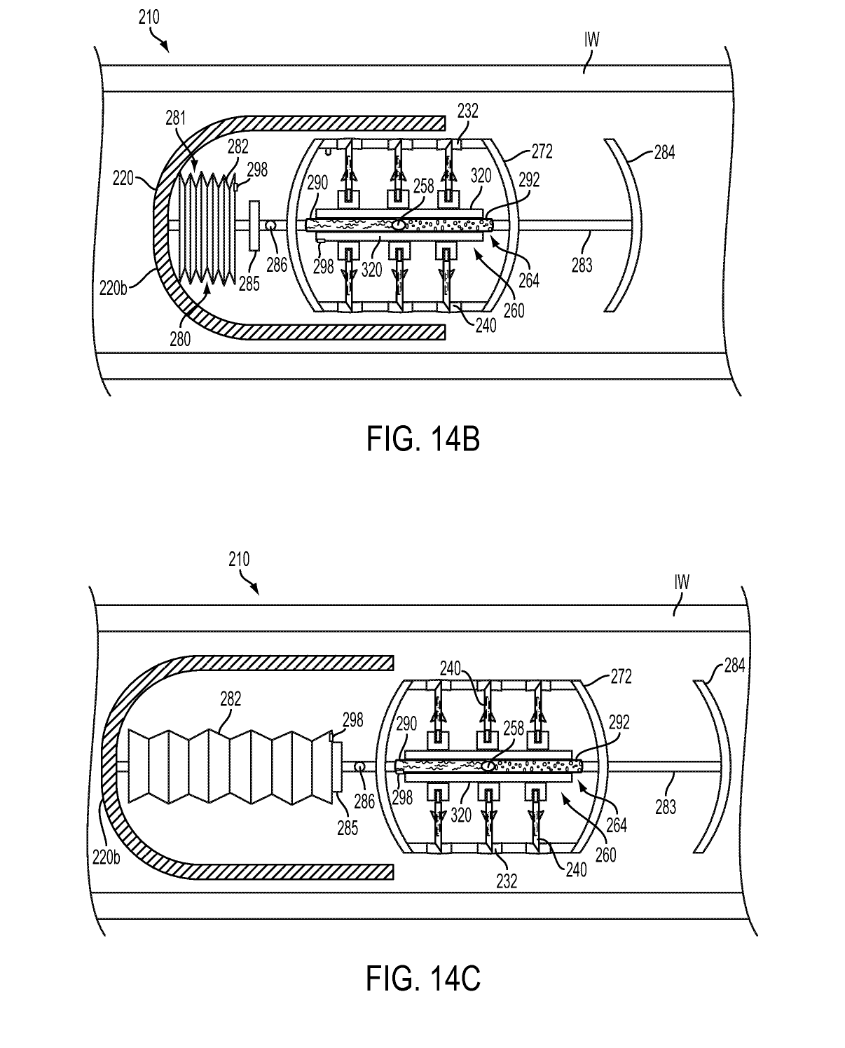

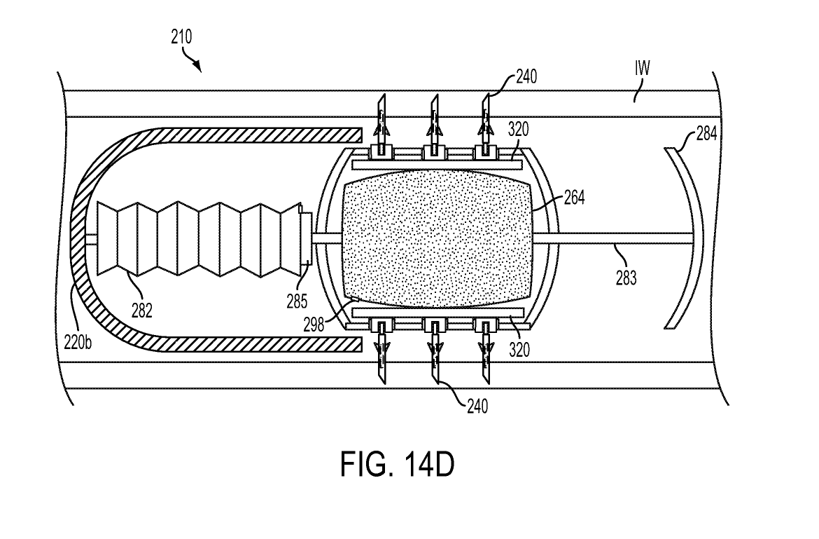

A further embodiment of device 210 is shown in FIGS. 14A-14D. Device 210 comprises a capsule 220 sized to be swallowed and pass through the intestinal tract, a delivery assembly, including one or more tissue penetrating members 240 and an actuator 250, and an extender assembly 280. The extender assembly 280 is configured to align the capsule with the intestine so that tissue penetrating members 250 properly pierce the tissue of the intestinal wall IW. Typically, this will entail aligning a longitudinal axis of the capsule with a longitudinal axis of the intestine; however, other alignments are also contemplated. The actuator 250 is configured to deliver functionalized particles 100 into the intestinal wall and includes an expandable member 260.

Extender assembly 280 comprises an expandable member 281, a track 283, a lead 284 and a stop 285. Expandable member 281 may be provided as a balloon 282 or any other expandable device discussed herein or known in the art. Lead 284 may generally be shape as a rounded structure so as to gently align device 10 within the lumen of the intestine and may also be provided as a balloon. Delivery assembly 270, which may include a housing 272 for supporting actuator 250 and tissue penetrating members 240, is fixedly disposed on track 283. Housing 272 may include apertures 230 therein for passage of tissue penetrating members. In some examples, housing 272 may include guides 232 (not shown). Both lead 284 and stop 285 are also fixedly connected to track 283, all or a portion of which may be telescoping so as to extend in length.

In its expanded or deployed state, expandable member 281, which may be a balloon 282, extends the length of capsule 220 such that forces exerted by the peristaltic contractions of the small intestine SI on capsule 220 serve to align the longitudinal axis of the capsule 220 in a parallel fashion with the longitudinal axis of the small intestine SI. This in turn serves to align tissue penetrating members 220 in a perpendicular fashion with the surface of the intestinal wall IW to enhance and optimize the penetration of tissue penetrating members 240 into the intestinal wall IW. In addition to serving to align capsule 220 in the small intestine, extender assembly 280 is also configured to push delivery assembly 270 out of the capsule 220 prior to inflation of delivery balloon 264 (as is shown in FIG. 14c) so that the delivery assembly 270 is not encumbered by the capsule. In use, this push out function of extender assembly 280 improves the reliability for delivery of the therapeutic agent since it is not necessary to wait for particular portions of the capsule (e.g., those overlying the delivery mechanism) to be degraded before drug delivery can occur. In some examples, balloon 282 may inflate to have a length in a range between about one-and-a-half to two times the length of the capsule 220 before inflation of balloon 282.

Capsule 220 may be fabricated in two parts a first segment 220a and a second segment 220b. First and second segments 220a and 220b are configured to degrade upon reaching the target intestinal region and separate from one another, exposing the internal components of the device 210. In some embodiments, such as is shown in FIG. 14B, only segment 220a will initially degrade and break away. Upon ingress of intestinal fluids into the interior of the device 210, balloon 282 will expand in accordance with methods described above, as shown in FIG. 14C. For example, balloon 282 may be formed with two compartments, separated by a degradable release, each containing reactive substances designed to create a gas when mixed. In other examples, balloon 282 may expand in response to a chemical, electrical, mechanical or external stimulus. As it expands, balloon 282 applies force at one end on stop 285 and at the other end on the internal surface of the capsule 220 (or, if capsule 220 is designed to fully degrade, on another stop), causing track 283 to extend thereby pushing delivery assembly 270 out and away from the capsule second segment 220b.

Actuator 250 is designed not to advance tissue penetrating members 240 into the intestinal wall until extender assembly 280 has extended and pushed delivery assembly away from the capsule second segment 220b. In the embodiments shown in FIGS. 14A-14D, actuator 250 includes an expandable member 260 in the form of a balloon 264. Similar to the embodiments described above, in one embodiment, balloon 264 may have a first compartment 290 containing a liquid 294, separated from a second compartment 292 containing one or more reactants 296 by a release 258. Release 258 may degrade upon contact with intestinal fluids F, allowing liquid 294 to mix with reactants 296 to produce a gas 298, thereby expanding balloon 264. Release 258 is configured to degrade at a rate such that first and second compartments are permitted to mix after enough time has passed for balloon 282 to expand and extend delivery assembly 270. Expansion of balloon 264, as described herein, acts upon platforms 120 pushing delivery members 240 out through apertures 230 and into intestinal wall IW, thereby delivering functionalized particles 100, as shown in FIG. 14D.

In another embodiment, balloon 264 may be fluidically coupled to balloon 282 via a lumen in track 283. The two balloons may be separated by a valve 286 designed to fail or otherwise allow air to pass therethrough once balloon 282 is fully or substantially expanded. In use, valve 286 allows for a sequenced inflation of balloon 282 and 264 such that balloon 282 is fully or otherwise substantially inflated before balloon 264 is inflated. This, in turn, allows balloon 282 to push balloon 264 along with the rest of delivery assembly 270out of capsule 220 before balloon 264 inflates so that deployment of tissue penetrating members 240 is not obstructed by capsule 220. In use, such an approach improves the reliability of the penetration of tissue penetrating members 240 into intestinal wall IW both in terms of achieving a desired penetration depth and delivering greater numbers of the penetrating members 240 contained in capsule 220.

One or both of balloons 264 and 282 may also include a deflation valve 298 which serves to deflate the balloon after inflation. Similar mechanisms and variations of deflation valve 298 may be used as were described above. Additionally, as further backup for insured deflation, one or more puncture elements 310 can be attached to the inside surface of the housing such that when balloon 264 fully inflates, it contacts the puncture element 310 and is deflated.

In some embodiments, one or more components of device 10 can be packed inside capsule 220 in a folded, furled or other desired configuration to conserve space within the interior volume 234 of the capsule. Folding can be done using preformed creases or other folding feature or method known in the arts. In particular embodiments, folding balloons 264 and 282 can also act to ensure that the balloons inflate correctly, with the desired orientation and in the desired sequence.

Tissue penetrating members 240, platforms 320 and connectors 252 can be positioned on one or multiple faces of balloon 264. For example, as shown in FIG. 14A, delivery assembly 270 can include tissue penetrating members 240 on opposite faces of balloon 264, so as to provide a substantially equal distribution of force to opposite sides of the intestinal wall IW upon expansion of balloon 264. The platforms 320, connectors 252 or penetrating members 240 themselves may be attached to the balloon surface using adhesives or other joining methods known in the arts.

In other variations of device 10, in addition or as an alternative to use of an expandable member 60, actuator 50 can also comprise an electro-mechanical device/mechanism such as a solenoid, or a piezoelectric device. In one embodiment, a piezoelectric actuator can comprise a shaped piezoelectric element which has a non-deployed and deployed state. This element can be configured to go into the deployed state upon the application of a voltage and then return to the non-deployed state upon the removal of the voltage. This may allow for a reciprocating motion of the actuator so as to both advance the tissue penetrating member and then withdraw it. The voltage for the piezoelectric element can be generated using a battery or a piezoelectric based energy converter which generates voltage by mechanical deformation such as that which occurs from compression of the capsule 20 by a peristaltic contraction of the small intestine around the capsule. In one embodiment, deployment of tissue penetrating members 40 can in fact be triggered from a peristaltic contraction of the small intestine which provides the mechanical energy for generating voltage for the piezoelectric element.

Further, as an alternative or supplement to internally activated delivery, in some embodiments, the user may externally send a signal to a release 58 or directly to actuator 50 to activate the actuator 50 to deliver functionalized particles 100. This may be achieved by means of RF, magnetic or other wireless signaling means known in the art, such as by use of a controllable valve for example, a radio frequency (RF) controlled miniature solenoid valve or other electro-mechanical control valve (not shown). In other embodiments, release 58 may comprise a controllable isolation valve provided as a miniature magnetically controlled valve such as a magnetically controlled miniature reed switch (not shown). Such electromechanical or magnetic-based valves can be fabricated using MEMS and other micro-manufacturing methods. In these and related embodiments, the user can use an external reader, such as a handheld communication device, mobile device, handheld computer, or other computing device to send and receive signals from device 10.

In such embodiments, swallowable device may also include a transmitter 29 such as an RF transceiver chip or other like communication device/circuitry. External reader may include a signaling means and also a means for informing the user when device 10 is in the small intestine or other location in the GI tract, such as a user interface or display. The external reader can also be configured to alert the user when actuator 50 has been activated and the selected functionalized particles 100 delivered. Such confirmation may allow the user to take other appropriate actions, such as eating a meal, taking a particular drug or therapeutic agent, take a rest, etc. functionalized particles/therapeutic agents as well as make other related decisions (e.g., for diabetics to eat a meal or not and what foods should be eaten). The handheld device can also be configured to send a signal to swallowable device 10 to over-ride release 58 or actuator 50 thereby allowing the user to intervene to prevent, delay or accelerate the delivery of functionalized particles, based upon other symptoms and/or patient actions (e.g., eating a meal, deciding to go to sleep, exercise etc.). The user may also externally trigger release 58 or activate actuator 60 at a selected time period after swallowing the capsule. The time period can be correlated to a typical transit time or range of transit times for food moving through the user's GI tract to a particular location in the tract such as the small intestine.

Other embodiments and configurations of a device for delivering functionalized particles to the intestinal tract are also contemplated. For example, other swallowable capsule-like delivery devices may also be used herein. Swallowable capsules may utilize certain enteric, protective or sustained-release coatings, such as Eudragit®, which can be configured to dissolve in the intestines, but not in low pH environment of the stomach. Embodiments of device using such coatings or materials may be configured such that the capsule dissolves in the intestine, delivering the functionalized particles to the lumen of the intestine for subsequent diffusion into the tissue and blood.

In other examples, a patch-like device may also be used to deliver functionalized particles to the intestinal tract. The patch may be delivered in a swallowable conformation or as part of a swallowable device and be configured to deploy at a target location in the intestinal tract. Alternatively, the patch may be implanted directly on the surface of the intestinal wall. The patch may include a surface designed to adhere to or otherwise stay in close proximity to the intestinal wall so that the functionalized particles it carries may be delivered by diffusion into the tissue. In some examples, the functionalized particles may be disposed on or in the patch such that the particles may diffuse directly from the surface of the patch to the intestinal wall. For example, the patch may be fabricated in multiple layers with, for example, a layer of functionalized particles disposed between two layers of biodegradable materials. In other examples, the functionalized particles may be contained within a compartment within the patch designed to release the functionalized particles at the surface of the intestinal wall to be diffused into the tissue or blood. The patch may be formed from materials designed to withstand degradation in the stomach and dissolve in the intestinal tract.

### IV. EXAMPLE SYSTEMS

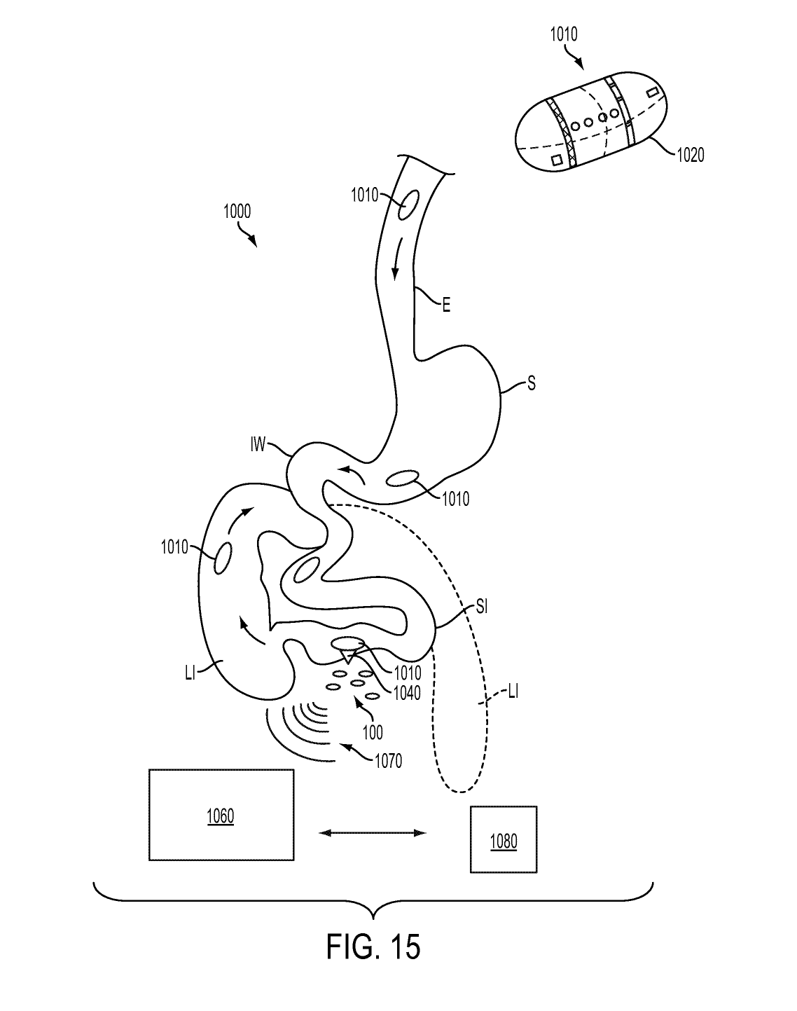

A system 1000, including a swallowable device 1010 comprising a capsule 1020, at least one tissue penetrating member 1040, and an actuator 1050 (not shown), as shown in FIG. 15, may also be provided. Swallowable device 1010 may include any embodiments of the swallowable devices described above. The system may further include a plurality of functionalized particles 100 configured to interact with one or more target analytes present in blood in a lumen of the subsurface vasculature, disposed within a capsule 1020 of the device 1010. The plurality of physiological parameters obtained by the functionalized particles 100 delivered by an embodiment of the swallowable device 1010 may be measured by a detector 1060 configured to detect an analyte response signal 1070 transmitted form the portion of subsurface vasculature. The analyte response signal 1070 may be related to the interaction of the one or more target analytes to the functionalized particles 100. In further embodiments, the system 1000 may also include a processor 1080 configured to detect the presence or absence of the clinically-relevant analyte based, at least in part, on the analyte response signal 1070. The processor 1080 may also be configured, in other examples, to determine a concentration of the clinically-relevant analyte based, at least in part, on the analyte response signal 1070. The detector 1060 may be located internal or external to the body of the patient and may be provided on a single platform with the processor 1080. In other examples, the processor 1080 may be remote from the detector 1060.

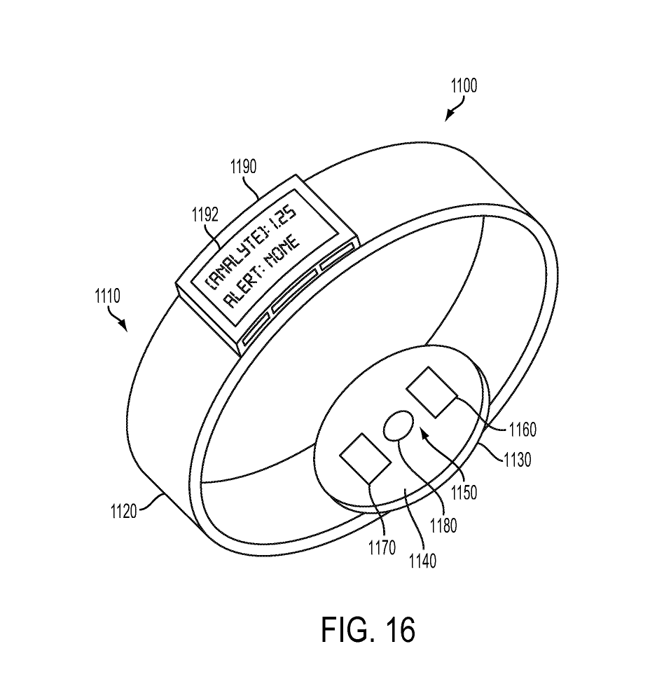

In some examples, the detector may be mounted on a wearable device 1100 configured to automatically measure a plurality of physiological parameters of a person wearing the device. The term “wearable device,” as used in this disclosure, refers to any device that is capable of being worn at, on or in proximity to a body surface, such as a wrist, ankle, waist, chest, or other body part. In order to take in vivo measurements in a noninvasive manner from outside of the body, the wearable device may be positioned on a portion of the body where subsurface vasculature is easily observable, the qualification of which will depend on the type of detection system used. The device may be placed in close proximity to the skin or tissue, but need not be touching or in intimate contact therewith. A mount 1110, such as a belt, wristband, ankle band, etc. can be provided to mount the device at, on or in proximity to the body surface. The mount 1110 may prevent the wearable device from moving relative to the body to reduce measurement error and noise. In one example, shown in FIG. 16, the mount 1110, may take the form of a strap or band 120 that can be worn around a part of the body. Further, the mount 1110 may be an adhesive substrate for adhering the wearable device 1100 to the body of a wearer.

A measurement platform 1130 is disposed on the mount 1110 such that it can be positioned on the body where subsurface vasculature is easily observable. An inner face 1140 of the measurement platform is intended to be mounted facing to the body surface. The measurement platform 1130 may house the data collection system 1150, which may include at least one detector 1160 for detecting at least one physiological parameter, which could include any parameters that may relate to the health of the person wearing the wearable device. For example, the detector 1160 could be configured to measure blood pressure, pulse rate, respiration rate, skin temperature, etc. At least one of the detectors 1160 is configured to noninvasively measure one or more analytes in blood circulating in subsurface vasculature proximate to the wearable device. In a non-exhaustive list, detector 1160 may include any one of an optical (e.g., CMOS, CCD, photodiode), acoustic (e.g., piezoelectric, piezoceramic), electrochemical (voltage, impedance), thermal, mechanical (e.g., pressure, strain), magnetic, or electromagnetic (e.g., magnetic resonance) sensor. The components of the data collection system 150 may be miniaturized so that the wearable device may be worn on the body without significantly interfering with the wearer's usual activities.

In some examples, the data collection system 1150 further includes a signal source 1170 for transmitting an interrogating signal that can penetrate the wearer's skin into the portion of subsurface vasculature, for example, into a lumen of the subsurface vasculature. The interrogating signal can be any kind of signal that is benign to the wearer, such as electromagnetic, magnetic, optic, acoustic, thermal, mechanical, and results in a response signal that can be used to measure a physiological parameter or, more particularly, that can detect the binding of the clinically-relevant analyte to the functionalized particles. In one example, the interrogating signal is an electromagnetic pulse (e.g., a radio frequency (RF) pulse) and the response signal is a magnetic resonance signal, such as nuclear magnetic resonance (NMR). In another example, the interrogating signal is a time-varying magnetic field, and the response signal is an externally-detectable physical motion due to the time-varying magnetic field. The time-varying magnetic field modulates the particles by physical motion in a manner different from the background, making them easier to detect. In a further example, the interrogating signal is an electromagnetic radiation signal. In particular, the interrogating signal may be electromagnetic radiation having a wavelength between about 400 nanometers and about 1600 nanometers. The interrogating signal may, more particularly, comprise electromagnetic radiation having a wavelength between about 500 nanometers and about 1000 nanometers. In some examples, the functionalized particles include a fluorophore. The interrogating signal may therefore be an electromagnetic radiation signal with a wavelength that can excite the fluorophore and penetrate the skin or other tissue and subsurface vasculature (e.g., a wavelength in the range of about 500 to about 1000 nanometers), and the response signal is fluorescence radiation from the fluorophore that can penetrate the subsurface vasculature and tissue to reach the detector.

In some cases, an interrogating signal is not necessary to measure one or more of the physiological parameters and, therefore, the wearable device 1100 may not include a signal source 1170. For example, the functionalized particles may include an autofluorescent or luminescent marker, such as a fluorophore, that will automatically emit a response signal indicative of the binding of the clinically-relevant analyte to the functionalized particles, without the need for an interrogating signal or other external stimulus. In some examples, the functionalized particles may include a chemiluminescent marker configured to produce a response signal in the form of fluorescence radiation produced in response to a chemical reaction initiated, at least in part, to the binding of the target analyte to the particle.

A collection magnet 1180 may also be included in the data collection system 1150. In such embodiments, the functionalized particles may also be made of or be functionalized with magnetic materials, such as ferromagnetic, paramagnetic, super-paramagnetic, or any other material that responds to a magnetic field. The collection magnet 180 is configured to direct a magnetic field into the portion of subsurface vasculature that is sufficient to cause functionalized magnetic particles to collect in a lumen of that portion of subsurface vasculature. The magnet may be an electromagnet that may be turned on during measurement periods and turned off when a measurement period is complete so as to allow the magnetic particles to disperse through the vasculature.

The wearable device 1100 may also include a user interface 1190 via which the wearer of the device may receive one or more recommendations or alerts generated either from a remote server or other remote computing device, or from a processor within the device. The alerts could be any indication that can be noticed by the person wearing the wearable device. For example, the alert could include a visual component (e.g., textual or graphical information on a display), an auditory component (e.g., an alarm sound), and/or tactile component (e.g., a vibration). Further, the user interface 1190 may include a display 1192 where a visual indication of the alert or recommendation may be displayed. The display 1192 may further be configured to provide an indication of the measured physiological parameters, for instance, the concentrations of certain blood analytes being measured.

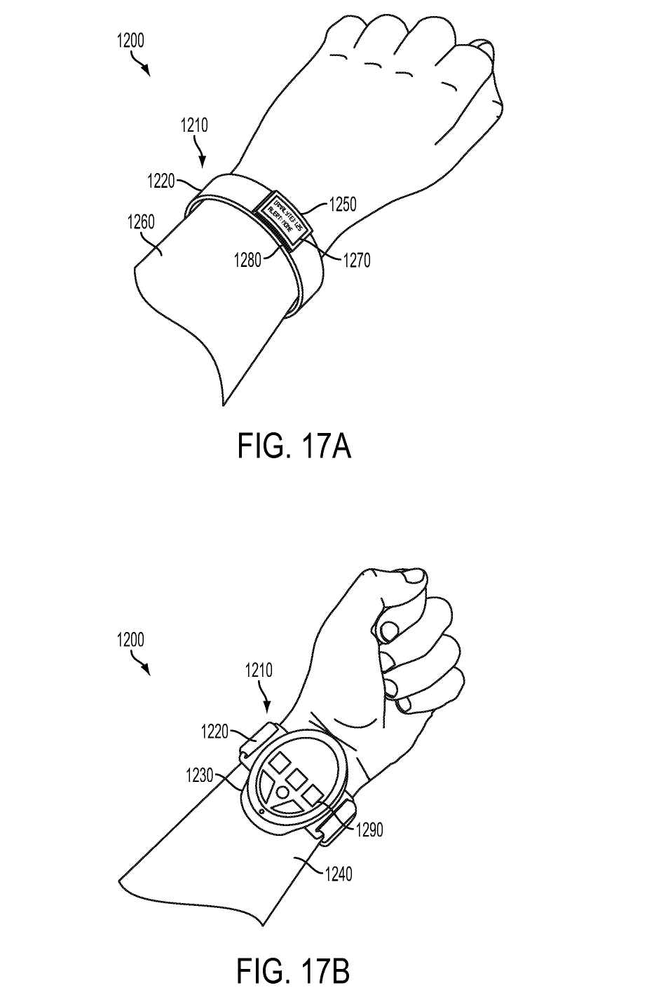

In one example, the wearable device is provided as a wrist-mounted device 1200, as shown in FIGS. 17A and 17B. The wrist-mounted device may be mounted to the wrist of a living subject with a wristband or cuff, similar to a watch or bracelet. As shown in FIGS. 2A and 2B, the wrist mounted device 1200 may include a mount 1210 in the form of a wristband 1220, a measurement platform 1230 positioned on the anterior side 1240 of the wearer's wrist, and a user interface 1250 positioned on the posterior side 1260 of the wearer's wrist. The wearer of the device may receive, via the user interface 1250, one or more recommendations or alerts generated either from a remote server or other remote computing device, or alerts from the measurement platform. Such a configuration may be perceived as natural for the wearer of the device in that it is common for the posterior side 1260 of the wrist to be observed, such as the act of checking a wrist-watch. Accordingly, the wearer may easily view a display 1270 on the user interface. Further, the measurement platform 1230 may be located on the anterior side 1240 of the wearer's wrist where the subsurface vasculature may be readily observable. However, other configurations are contemplated.

The display 1270 may be configured to display a visual indication of the alert or recommendation and/or an indication of the measured physiological parameters, for instance, the concentrations of certain blood analytes being measured. Further, the user interface 1250 may include one or more buttons 1280 for accepting inputs from the wearer. For example, the buttons 1280 may be configured to change the text or other information visible on the display 270. As shown in FIG. 17B, measurement platform 1230 may also include one or more buttons 1290 for accepting inputs from the wearer. The buttons 1290 may be configured to accept inputs for controlling aspects of the data collection system, such as initiating a measurement period, or inputs indicating the wearer's current health state (i.e., normal, migraine, shortness of breath, heart attack, fever, “flu-like” symptoms, food poisoning, etc.).

In other examples of wrist-mounted device, the measurement platform and user interface may both be provided on the same side of the wearer's wrist, in particular, the anterior side of the wrist. The wrist mounted device may also be provided with a watch face on the posterior side of the wearer's wrist.

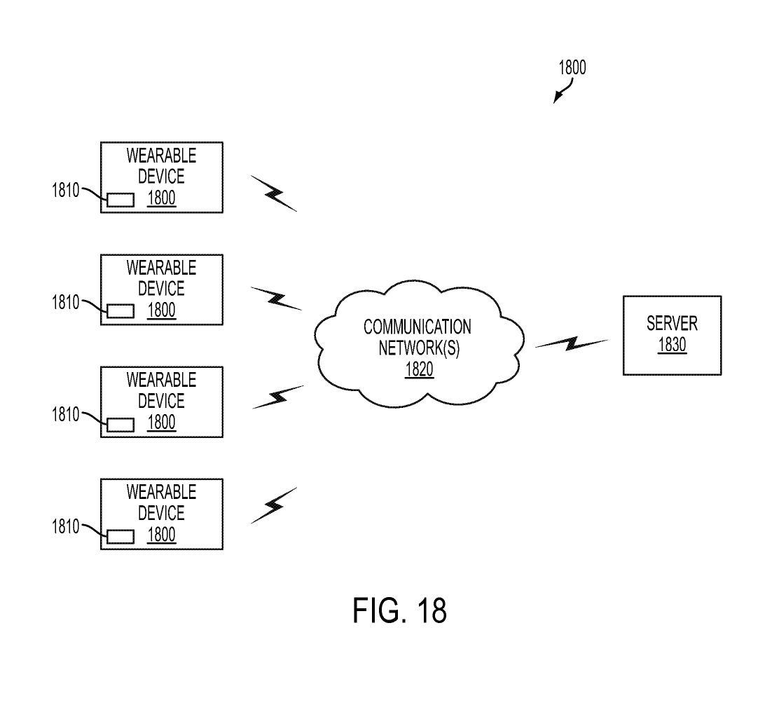

FIG. 18 is a simplified schematic of a system including one or more wearable devices 1800. The one or more wearable devices 700 may be configured to transmit data via a communication interface 1810 over one or more communication networks 1820 to a remote server 1830. In one embodiment, the communication interface 1810 includes a wireless transceiver for sending and receiving communications to and from the server 1830. In further embodiments, the communication interface 1810 may include any means for the transfer of data, including both wired and wireless communications. For example, the communication interface may include a universal serial bus (USB) interface or a secure digital (SD) card interface. Communication networks 1820 may be any one of may be one of: a plain old telephone service (POTS) network, a cellular network, a fiber network and a data network. The server 1830 may include any type of remote computing device or remote cloud computing network. Further, communication network 1820 may include one or more intermediaries, including, for example wherein the wearable device 1800 transmits data to a mobile phone or other personal computing device, which in turn transmits the data to the server 1830.

In addition to receiving communications from the wearable device 1800, such as collected physiological parameter data and data regarding health state as input by the user, the server may also be configured to gather and/or receive either from the wearable device 1800 or from some other source, information regarding a wearer's overall medical history, environmental factors and geographical data. For example, a user account may be established on the server for every wearer that contains the wearer's medical history. Moreover, in some examples, the server 1830 may be configured to regularly receive information from sources of environmental data, such as viral illness or food poisoning outbreak data from the Centers for Disease Control (CDC) and weather, pollution and allergen data from the National Weather Service. Further, the server may be configured to receive data regarding a wearer's health state from a hospital or physician. Such information may be used in the server's decision-making process, such as recognizing correlations and in generating clinical protocols.

Additionally, the server may be configured to gather and/or receive the date, time of day and geographical location of each wearer of the device during each measurement period. Such information may be used to detect and monitor spatial and temporal spreading of diseases. As such, the wearable device may be configured to determine and/or provide an indication of its own location. For example, a wearable device may include a GPS system so that it can include GPS location information (e.g., GPS coordinates) in a communication to the server. As another example, a wearable device may use a technique that involves triangulation (e.g., between base stations in a cellular network) to determine its location. Other location-determination techniques are also possible.

The server may also be configured to make determinations regarding the efficacy of a drug or other treatment based on information regarding the drugs or other treatments received by a wearer of the device and, at least in part, the physiological parameter data and the indicated health state of the user. From this information, the server may be configured to derive an indication of the effectiveness of the drug or treatment. For example, if a drug is intended to treat nausea and the wearer of the device does not indicate that he or she is experiencing nausea after beginning a course of treatment with the drug, the server may be configured to derive an indication that the drug is effective for that wearer. In another example, a wearable device may be configured to measure blood glucose. If a wearer is prescribed a drug intended to treat diabetes, but the server receives data from the wearable device indicating that the wearer's blood glucose has been increasing over a certain number of measurement periods, the server may be configured to derive an indication that the drug is not effective for its intended purpose for this wearer.

Further, some embodiments of the system may include privacy controls which may be automatically implemented or controlled by the wearer of the device. For example, where a wearer's collected physiological parameter data and health state data are uploaded to a cloud computing network for trend analysis by a clinician, the data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined.

Additionally or alternatively, wearers of a device may be provided with an opportunity to control whether or how the device collects information about the wearer (e.g., information about a user's medical history, social actions or activities, profession, a user's preferences, or a user's current location), or to control how such information may be used. Thus, the wearer may have control over how information is collected about him or her and used by a clinician or physician or other user of the data. For example, a wearer may elect that data, such as health state and physiological parameters, collected from his or her device may only be used for generating an individual baseline and recommendations in response to collection and comparison of his or her own data and may not be used in generating a population baseline or for use in population correlation studies.

### IV. ILLUSTRATIVE METHODS

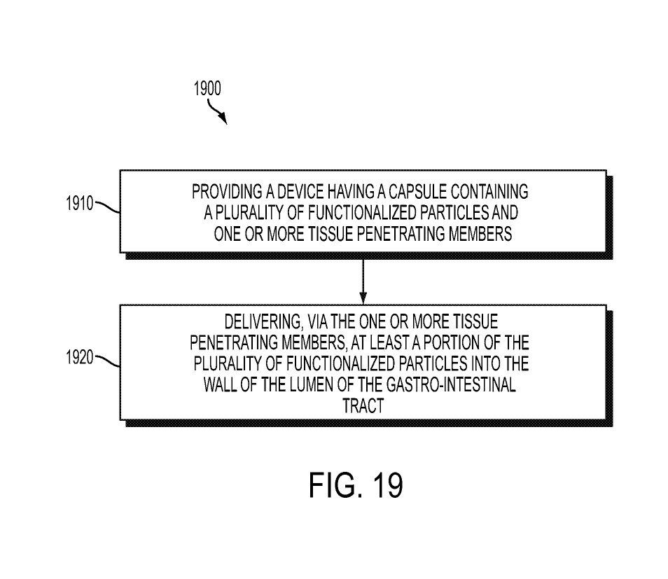

FIG. 19 is a flowchart of a method 1900 for delivering functionalized particles to the body. A swallowable device having: (1) a capsule containing a plurality of functionalized particles, and (2) one or more tissue penetrating members is first ingested (1910). The capsule is sized to pass through a lumen of a gastrointestinal tract. Each of the one or more tissue penetrating members has a lumen and an exit through which the functionalized particles can pass. The tissue penetrating members are further configured to puncture a wall of the lumen of the gastrointestinal tract. At least a portion of the plurality of functionalized particles are delivered via the one or more tissue penetrating members into the wall of the lumen of the gastrointestinal tract (1920). Delivery of the functionalized particles may occur in response to a chemical condition in the gastrointestinal tract. For example, delivery may occur upon exposure to chemical conditions in the small or large intestine such as pH, such as upon exposure to a selected pH in the small intestine, e.g., 7.0, 7.1, 7.2, 7.3, 7.4, 7.5, 7.6 8.0 or greater. In some examples, delivery may occur in a pH range from 7.0 to 7.5. In other examples, delivery may occur in response to a mechanical or electrochemical stimulus. In still further examples, delivery may occur in response to a stimulus remote from the swallowable device.

In another example method, a plurality of functionalized particles is loaded into a device having a capsule sized to pass through a lumen of a gastrointestinal tract, one or more tissue penetrating members, and an actuator having a first configuration and a second configuration. The plurality of functionalized particles may be loaded into the capsule such that they are communication with the one or more tissue penetrating members. The one or more tissue penetrating members may be configured to puncture a wall of the lumen of the intestinal tract and each may have a respective penetrating-member exit. The actuator is configured to retain the one or more tissue penetrating members within the capsule in the first configuration. Further, by transitioning from the first configuration to the second configuration, the actuator is configured to advance the one or more tissue penetrating members from the capsule into a wall of the lumen of the gastrointestinal tract. At least a portion of the functionalized particles may be delivered into the wall of the lumen of the gastrointestinal tract by the actuator transitioning from the first configuration to the second configuration. The actuator may be configured to transition from the first configuration to the second configuration in response to a chemical condition in the gastrointestinal tract, such as a predetermined pH value, in response to a mechanical input, or in response to an input remote from the device.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

### V. CONCLUSION

Where example embodiments involve information related to a person or a device of a person, some embodiments may include privacy controls. Such privacy controls may include, at least, anonymization of device identifiers, transparency and user controls, including functionality that would enable users to modify or delete information relating to the user's use of a product.

Further, in situations in where embodiments discussed herein collect personal information about users, or may make use of personal information, the users may be provided with an opportunity to control whether programs or features collect user information (e.g., information about a user's medical history, social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

What is claimed is:

1. A device comprising:
a capsule sized to pass through a lumen of a gastrointestinal tract;
a plurality of functionalized particles disposed within the capsule;
one or more tissue penetrating members configured to puncture a wall of the lumen of the intestinal tract; and
an actuator having a first configuration and a second configuration, wherein the actuator is configured to retain the plurality of functionalized particles within the capsule in the first configuration, and wherein the actuator is configured to advance the plurality of functionalized particles from the capsule into a wall of the lumen of the gastrointestinal tract via the one or more tissue penetrating members by the actuator transitioning from the first configuration to the second configuration.

2. The device of claim 1, further comprising a release configured to releasably maintain the actuator in the first configuration.

3. The device of claim 2, wherein the release is configured to degrade in response to a chemical condition in the gastrointestinal tract, such that degradation of the release causes the actuator to transition from the first configuration to the second configuration.

4. The device of claim 3, wherein the chemical condition comprises a predetermined pH value.

5. The device of claim 3, wherein the actuator comprises a spring.

6. The device of claim 5, wherein the release is coupled to the spring such that the release retains the spring in a compressed state in the first configuration and degradation of the release causes the spring to be released.

7. The device of claim 3, wherein the actuator comprises a balloon.

8. The device of claim 7, wherein, in the first configuration of the actuator, the release is coupled to the balloon to define a first compartment separated from a second compartment.

9. The device of claim 8, wherein the first compartment and the second compartment contain a first reagent and a second reagent, respectively, that are configured to produce a gas when mixed together.

10. The device of claim 9, wherein the release is configured such that degradation of the release causes the first and second reagents to mix together, and wherein the actuator transitioning from the first configuration to the second configuration comprises the balloon expanding in response to the gas produced by the mixing of the first and second reagents.

11. The device of claim 1, wherein each of the one of the one or more tissue penetrating members comprises a respective penetrating-member lumen and respective penetrating-member exit through which the functionalized particles can pass.

12. The device of claim 11, wherein each of the one of the one or more tissue penetrating members further comprises a respective delivery member coupled to the actuator and configured to advance the functionalized particles through the respective penetrating-member lumen toward the respective penetrating-member exit.

13. The device of claim 1, wherein the functionalized particles include a receptor having an affinity for a target analyte.

14. The device of claim 13, wherein the receptor is chosen from the group consisting of antibodies, nucleic acids, low molecular weight ligands, peptides, proteins, polysaccharides, polyunsaturated fatty acids, plasmids, viruses and phages.

15. The device of claim 1, wherein the functionalized particles include one or more of a fluorescent, an autofluorescent, a luminescent and a chemiluminescent marker.

16. The device of claim 1, wherein the functionalized particles have a shape chosen from the group consisting of sphere, rod, flake, disc, diamond, and non-symmetrical.

17. The device of claim 1, wherein the functionalized particles include a paramagnetic, super-paramagnetic or ferromagnetic material.

18. The device of claim 1, wherein the functionalized particles are formed from a biodegradable material.

19. A method, comprising:
providing a device having:
a capsule containing a plurality of functionalized particles, wherein the capsule is sized to pass through a lumen of a gastrointestinal tract; and
one or more tissue penetrating members configured to puncture a wall of the lumen of the gastrointestinal tract, each of the one or more tissue penetrating members having a respective penetrating-member lumen and penetrating-member exit through which the functionalized particles can pass;
said device configured to deliver, via the one or more tissue penetrating members, at least a portion of the plurality of functionalized particles into the wall of the lumen of the gastrointestinal tract.

20. The method of claim 19, wherein the device is configured to deliver at least a portion of the functionalized particles in response to a chemical condition in the gastrointestinal tract.

21. The method of claim 20, wherein the condition comprises a predetermined pH value.

22. The method of claim 19, wherein the device is configured to deliver at least a portion of the functionalized particles in response to a mechanical input.

23. The method of claim 19, wherein the device is configured to deliver at least a portion of the functionalized particles in response to an input remote from the device.

24. A system comprising:
a swallowable device comprising:
a capsule sized to pass through a lumen of a gastrointestinal tract;
one or more tissue penetrating members configured to puncture a wall of the lumen of the intestinal tract; and
an actuator having a first configuration and a second configuration;
a plurality of functionalized particles disposed within the capsule, the functionalized particles configured to interact with one or more target analytes present in blood in a lumen of subsurface vasculature;
wherein the actuator is configured to retain the plurality of functionalized particles within the capsule in the first configuration, and wherein the actuator is configured to advance the plurality of functionalized particles from the capsule into a wall of the lumen of the gastrointestinal tract via the one or more tissue penetrating members by the actuator transitioning from the first configuration to the second configuration; and
a detector configured to detect an analyte response signal transmitted from the portion of subsurface vasculature, wherein the analyte response signal is related to the interaction of the one or more target analytes with the functionalized particles.

25. The system of claim 24, further comprising a wearable device having a mount configured to mount the wearable device to an external body surface proximate to a portion of subsurface vasculature, said detector mounted on said wearable device.

26. The system of claim 24, further comprising a processor configured to detect the presence or absence of the clinically-relevant analyte based on the analyte response signal.

27. The system of claim 26, wherein the processor is further configured to determine a concentration of the clinically-relevant analyte based on the analyte response signal.

28. The system of claim 24, wherein the actuator is a balloon.

29. A method, comprising:
loading a plurality of functionalized particles into a device having:
a capsule sized to pass through a lumen of a gastrointestinal tract;
one or more tissue penetrating members configured to puncture a wall of the lumen of the intestinal tract, each of the tissue penetrating members having a respective penetrating-member exit; and
an actuator having a first configuration and a second configuration, wherein the actuator is configured to retain the plurality of functionalized particles within the capsule in the first configuration, and wherein the actuator is configured to advance the plurality of functionalized particles from the capsule into a wall of the lumen of the gastrointestinal tract via the one or more tissue penetrating members by the actuator transitioning from the first configuration to the second configuration.

30. The method of claim 29, wherein the plurality of functionalized particles are loaded into the capsule in communication with the one or more tissue penetrating members.

31. The method of claim 29, wherein the actuator is configured to deliver, via the one or more tissue penetrating members, at least a portion of the plurality of functionalized particles into the wall of the lumen of the gastrointestinal tract by the actuator transitioning from the first configuration to the second configuration.

32. The method of claim 29, wherein the actuator is configured to transition from the first configuration to the second configuration in response to a chemical condition in the gastrointestinal tract.

33. The method of claim 31, wherein the condition comprises a predetermined pH value.

34. The method of claim 19, wherein the actuator is configured to transition from the first configuration to the second configuration in response to a mechanical input.

35. The method of claim 19, wherein the actuator is configured to transition from the first configuration to the second configuration in response to an input remote from the device.
